SEQUENCE LISTING (15 sequences)

SEQ ID NO: 1 - DNA, 1394 bp, Artificial Sequence
  Synthetic Polynucleotide
tccctatcagtgatagagattgacatccctatcagtgatagagatactgagcacatcagcaggacgcactgaccactttaagaaggagatataccatggccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaagtcgacaacctaggaaaaacctgaggaaaatgcatagctagaggcatcaaataaaacgaaaggctcagtcgaaagactgggcctttcgttttatctgttgtttgtcggtgaacgctctcctgagtaggacaaatccgccgggagcggatttgaacgctgcgaagcaacggcccggagggtggcgggcaggacgcccgccataaactgccaggcatcaaattaagcagaaggccatcctgacggatggcctttttgcgtttctacaaa

SEQ ID NO: 2 - DNA, 1394 bp, Artificial Sequence; identical to SEQ ID NO: 1
  Synthetic Polynucleotide

SEQ ID NO: 3 - DNA, 2711 bp, Artificial Sequence
  Synthetic Polynucleotide
tccctatcagtgatagagattgacatccctatcagtgatagagatactgagcacatcagcaggacgcactgaccactttaagaaggagatataccatggccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaataaaagcttctctagaaataattttgtttaactttaagaaggagatataccatgagccaatttgatatattatgtaaaacaccacctaaggtcctggttcgtcagtttgtggaaaggtttgaaagaccttcaggggaaaaaatagcatcatgtgctgctgaactaacctatttatgttggatgattactcataacggaacagcaatcaagagagccacattcatgagctataatactatcataagcaattcgctgagtttcgatattgtcaacaaatcactccagtttaaatacaagacgcaaaaagcaacaattctggaagcctcattaaagaaattaattcctgcttgggaatttacaattattccttacaatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacctcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaactctgaaccagtcctaaaacgagtaaataggaccggcaattcttcaagcaacaaacaggaataccaattattaaaagataacttagtcagatcgtacaacaaggctttgaagaaaaatgcgccttatccaatctttgctataaagaatggcccaaaatctcacattggaagacatttgatgacctcatttctgtcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaattaataagtcgacaacctaggaaaaacctgaggaaaatgcatagctagaggcatcaaataaaacgaaaggctcagtcgaaagactgggcctttcgttttatctgttgtttgtcggtgaacgctctcctgagtaggacaaatccgccgggagcggatttgaacgctgcgaagcaacggcccggagggtggcgggcaggacgcccgccataaactgccaggcatcaaattaagcagaaggccatcctgacggatggcctttttgcgtttctacaaa

SEQ ID NO: 4 - DNA, 1635 bp, Artificial Sequence
  Synthetic Polynucleotide
ataacttcgtataatgtatgctatacgaagttatgcagtttcatttgatgctcgatgagtttttctaagaattaattcatgagcggatacatatttgaatgtatttagaaaaataaacaaataggggttccgcgcacatttccccgaaaagtgccacctaggtatctggcactacgttcaggtaacctgaagctcgaatccagtactcgacgtctctagggcggcggatttgtcctactcaggagagcgttcaccgacaaacaacagataaaacgaaaggcccagtctttcgactgagcctttcgttttatttgatgcctctagcacgcgtacctggtggcgcgccttatttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtgtctaattttgaagttaactttgattccattcttttgtttgtctgccatgatgtatacattgtgtgagttatagttgtattccaatttgtgtccaagaatgtttccatcttctttaaaatcaataccttttaactcgattctattaacaagggtatcaccttcaaacttgacttcagcacgtgtcttgtagttcccgtcatctttgaaaaatatagttctttcctgtacataaccttcgggcatggcactcttgaaaaagtcatgctgtttcatatgatctgggtatctcgcaaagcattgaacaccataaccgaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttacgcatggtatatctccttcttaaagtggtcagtgcgtcctgctgatgtgctcagtatcttgttatccgctcacaatgtaaattgttatccgctcacaattgtatccgctcatgaattaattcttaggcatattcaaatcgttttcgttaccgcttgcaggcatcatgacagaacactacttcctataaacgctacacaggctcctgagattaataatgcggatctgtcccagactaataatcagaccgacgaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatagctagcataacttcgtatagcatacattatacgaagttat

SEQ ID NO: 5 - DNA, 1635 bp, Artificial Sequence; identical to SEQ ID NO: 4
  Synthetic Polynucleotide

SEQ ID NO: 6 - DNA, 1180 bp, Artificial Sequence
  Synthetic Polynucleotide
ataacttcgtataatgtatgctatacgaagttatgcagtttcatttgatgctcgatgaggaagttcctattctctagaaagtataggaacttcaagctcgaatccagtactcgacgtctctagggcggcggatttgtcctactcaggagagcgttcaccgacaaacaacagataaaacgaaaggcccagtctttcgactgagcctttcgttttatttgatgcctctagcacgcgtacctggtggcgcgccttatttgtatagttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtgtctaattttgaagttaactttgattccattcttttgtttgtctgccatgatgtatacattgtgtgagttatagttgtattccaatttgtgtccaagaatgtttccatcttctttaaaatcaataccttttaactcgattctattaacaagggtatcaccttcaaacttgacttcagcacgtgtcttgtagttcccgtcatctttgaaaaatatagttctttcctgtacataaccttcgggcatggcactcttgaaaaagtcatgctgtttcatatgatctgggtatctcgcaaagcattgaacaccataaccgaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttacgcatggtatatctccttcttaaagtggtcagtgcgtcctgctgatgtgctcagtatcttgttatccgctcacaatgtaaattgttatccgctcacaattgtatccgctcatgaattaattcttagaagttcctatactttctagagaataggaacttcaggcattgatggaatcgtagtctcaataacttcgtatagcatacattatacgaagttat

SEQ ID NO: 7 - DNA, 23 bp, Artificial Sequence
  Synthetic Polynucleotide
gacattaacctataaaaataggc

SEQ ID NO: 8 - DNA, 19 bp, Artificial Sequence
  Synthetic Polynucleotide
gcatcttccaggaaatctc

SEQ ID NO: 9 - DNA, 26 bp, Artificial Sequence
  Synthetic Polynucleotide
cccgtattgacgccgggcaagagcaa

SEQ ID NO: 10 - DNA, 784 bp, Artificial Sequence
  Synthetic Polynucleotide
ttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtgtctaattttgaagttaactttgattccattcttttgtttgtctgccatgatgtatacattgtgtgagttatagttgtattccaatttgtgtccaagaatgtttccatcttctttaaaatcaataccttttaactcgattctattaacaagggtatcaccttcaaacttgacttcagcacgtgtcttgtagttcccgtcatctttgaaaaatatagttctttcctgtacataaccttcgggcatggcactcttgaaaaagtcatgctgtttcatatgatctgggtatctcgcaaagcattgaacaccataaccgaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttacgcatggtatatctccttcttaaagtggtcagtgcgtcctgctgatgtgctcagtatcttgttatccgctcacaatgtaaattg

SEQ ID NO: 11 - DNA, 164 bp, Artificial Sequence
  Synthetic Polynucleotide
ataacttcgtataatgtatgctatacgaagttatgcagtttcatttgatgctcgatgagtttttctaagaattaattcatgagcggatacaattgtgagcggataacaatttacattgtgagcggataacaagatactgagcacatcagcaggacgcactgacc

SEQ ID NO: 12 - DNA, 214 bp, Artificial Sequence
  Synthetic Polynucleotide
ataacttcgtataatgtatgctatacgaagttatgcagtttcatttgatgctcgatgagtttttctaagaattaattcatgagcggatacatatttgaatgtatttagaaaaataaacaaataggggttccgcgcacatttccccgaaaagtgccacctaggtatctggcactacgttcaggtaacctgaagctcgaatccagtactcgacgtc

SEQ ID NO: 13 - DNA, 350 bp, Artificial Sequence
  Synthetic Polynucleotide
ataacttcgtataatgtatgctatacgaagttatgctagctatggagaaacagtagagagttgcgataaaaagcgtcaggtaggatccgctaatcttatggataaaaatgctatggcatagcaaagtgtgacgccgtgcaaataatcaatgtggacttttctgccgtgattatagacacttttgttacgcgtttttgtcatggctttggtcccgctttgttacagaatgcttttaataagcggggttaccggtttggttagcgagaagagccagtaaaagacgcagtgacggcaatgtctgatgcaatatggacaattggtttcttcgtcggtctgattattagtctggg

SEQ ID NO: 14 - DNA, 918 bp, Artificial Sequence
  Synthetic Polynucleotide
ttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtgtctaattttgaagttaactttgattccattcttttgtttgtctgccatgatgtatacattgtgtgagttatagttgtattccaatttgtgtccaagaatgtttccatcttctttaaaatcaataccttttaactcgattctattaacaagggtatcaccttcaaacttgacttcagcacgtgtcttgtagttcccgtcatctttgaaaaatatagttctttcctgtacataaccttcgggcatggcactcttgaaaaagtcatgctgtttcatatgatctgggtatctcgcaaagcattgaacaccataaccgaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttacgcatggtatatctccttcttaaagtggtcagtgcgtcctgctgatgtgctcagtatcttgttatccgctcacaatgtaaattgttatccgctcacaattgtatccgctcatgaattaattcttagaagttcctatactttctagagaataggaacttcaggcattgatggaatcgtagtctcaataacttcgtatagcatacattatacgaagttat

SEQ ID NO: 15 - DNA, 918 bp, Artificial Sequence
  Synthetic Polynucleotide
ttcatccatgccatgtgtaatcccagcagctgttacaaactcaagaaggaccatgtggtctctcttttcgttgggatctttcgaaagggcagattgtgtggacaggtaatggttgtctggtaaaaggacagggccatcgccaattggagtattttgttgataatggtctgctagttgaacgcttccatcttcaatgttgtgtctaattttgaagttaactttgattccattcttttgtttgtctgccatgatgtatacattgtgtgagttatagttgtattccaatttgtgtccaagaatgtttccatcttctttaaaatcaataccttttaactcgattctattaacaagggtatcaccttcaaacttgacttcagcacgtgtcttgtagttcccgtcatctttgaaaaatatagttctttcctgtacataaccttcgggcatggcactcttgaaaaagtcatgctgtttcatatgatctgggtatctcgcaaagcattgaacaccataaccgaaagtagtgacaagtgttggccatggaacaggtagttttccagtagtgcaaataaatttaagggtaagttttccgtatgttgcatcaccttcaccctctccactgacagaaaatttgtgcccattaacatcaccatctaattcaacaagaattgggacaactccagtgaaaagttcttctcctttacgcatggtatatctccttcttaaagtggtcagtgcgtcctgctgatgtgctcagtatcttgttatccgctcacaatgtaaattgttatccgctcacaattgtatccgctcatgaattaattcttagaagttcctatactttctagagaataggaacttcctcatcgagcatcaaatgaaactgcataacttcgtatagcatacattatacgaagttat

(19) United States
(12) Patent Application Publication
(10) Pub. No.: US 2017/0335334 A1
(43) Pub. Date: Nov. 23, 2017

(54) DNA CLOAKING TECHNOLOGIES

(71) Applicant: Massachusetts Institute of Technology, Cambridge, MA (US)

(72) Inventors: Timothy Kuan-Ta Lu, Cambridge, MA (US); Peter A. Carr, Medford, MA (US); Bijan Zakeri, Revere, MA (US)

(73) Assignee: Massachusetts Institute of Technology, Cambridge, MA (US)

(21) Appl. No.: 15/521,943

(22) PCT Filed: Oct. 29, 2015
(86) PCT No.: PCT/US15/58099

§ 371 Date: Apr. 26, 2017

Related U.S. Application Data
(60) Provisional application No. 62/072,113, filed on Oct. 29, 2014.

(51) Int. Cl. C12N 15/74; G06F 19/24; C12N 15/87; C12Q 1/68

(52) U.S. Cl. CPC C12N 15/74; C12Q 1/68; G06F 19/24; C12N 15/87; C12Q 2563/185; C12Q 2521/507

(57) ABSTRACT

The disclosure relates to the use of steganographic methods to camouflage information encoded on nucleic acids. Specifically, the method comprising preparing a pool of recombinant nucleic acid constructs, wherein at least one of the constnjcts comprises the nucleic acid sequence to be camouflaged and wherein the pool is heterogeneous with respect to the orientation of the nucleic acid sequence to be camouflaged, and the information is camouflaged using genetic recombination.

### RELATED APPLICATIONS

This application claims the benefit of U.S. provisional application serial number U.S. Ser. No. 62/072,113, filed on Oct. 29, 2014, and entitled “DNA Cloaking Technologies”, the entire content of which is incorporated herein by reference.

### FEDERALLY SPONSORED RESEARCH

This invention was made with government support under Contract No. N66001-12-C-4016 awarded by the Space and Naval Warfare Systems Center. The government has certain rights in the invention.

### BACKGROUND OF INVENTION

DNA synthesis and sequencing technologies have rapidly advanced over the past decades enabling facile storage and extraction of information from DNA. Consequently, synthetic DNA has gained additional functionality as a chemical storage medium that can harbor valuable data. Yet, bio-safeguards for protecting such data from sequencing by unauthorized users are currently lacking.

### SUMMARY OF INVENTION

In some aspects, the instant disclosure relates to the use of steganographic methods to camouflage information encoded on nucleic acids.

In some embodiments, the instant disclosure relates to a method for camouflaging a nucleic acid sequence comprising: preparing a pool of recombinant nucleic acid constructs, wherein at least one of the constructs comprises the nucleic acid sequence to be camouflaged and wherein the pool is heterogeneous with respect to the orientation of the nucleic acid sequence to be camouflaged. In some embodiments, the pool of constructs is obtained by nucleic acid synthesis. In some embodiments, the pool of constructs is maintained in a cell or cells. In some embodiments, the pool of constructs is maintained in a non-cellular environment.

In some embodiments, the instant disclosure relates to a method for camouflaging a nucleic acid sequence comprising: (a) preparing a recombinant nucleic acid construct that comprises the nucleic acid sequence to be camouflaged, wherein the nucleic acid sequence is flanked by opposing recognition sites of a bidirectional recombinase; (b) introducing the recombinant nucleic acid construct into a cell that comprises the bidirectional recombinase; (c) culturing the cell under conditions in which the cell multiplies to produce a plurality of cells; and, (d) creating a population of cells that is heterogeneous with respect to the orientation of the nucleic acid sequence by culturing the plurality of cells under conditions in which the bidirectional recombinase is expressed, whereby the nucleic acid sequence flanked by the opposing recognition sites of the bidirectional recombinase is inverted in a portion of the plurality of cells, thereby camouflaging the nucleic acid sequence.

In some embodiments, the instant disclosure relates to a method for camouflaging a nucleic acid sequence comprising: (a) preparing a recombinant nucleic acid construct that comprises the nucleic acid sequence to be camouflaged, wherein the nucleic acid sequence is flanked by opposing recognition sites of a bidirectional recombinase; (b) combining a plurality of the construct of (a) and a functional bidirectional recombinase in vitro, whereby the nucleic acid sequence flanked by the opposing recognition sites of the bidirectional recombinase is inverted, thereby producing a heterogeneous population of camouflaged constructs; and, (c) maintaining the heterogeneous population of (b) in a non-cellular environment.

In some embodiments, the nucleic acid comprises a protein-encoding gene. In some embodiments, the nucleic acid sequence is dispersed across a plurality of nucleic acids or is comprised of a plurality of segments. In some embodiments, at least two of the plurality of nucleic acids or the plurality of segments are flanked by opposing recognition sites of at least two different bidirectional recombinases, and wherein the cell or plurality of cells comprises the at least two different bidirectional recombinases.

In some embodiments of the method, the bidirectional recombinase(s) is/are expressed in the cell on one or more temperature sensitive plasmids. In some embodiments of the method, the bidirectional recombinase(s) is/are expressed constitutively in the plurality of cells. In some embodiments, the bidirectional recombinase(s) is/are expressed under control of one or more conditional promoters in the plurality of cells.

In some embodiments of the method, the cell is a prokaryotic cell. In some embodiments of the method, the cell is a eukaryotic cell. In some embodiments, the recombinant nucleic acid construct is integrated into the genome of the plurality of cells. In some embodiments, the cell(s) comprises at least one of Cre, Flp and R bidirectional recombinases and wherein the recognition sites flanking the nucleic acid sequence are operable with the at least one bidirectional recombinases and are selected from loxP, FRT and RS recognition sites. In some embodiments, the cell comprises two or more bidirectional recombinases and wherein the recognition sites flanking the nucleic acid are operable with the two or more bidirectional recombinases. In some embodiments, the cell(s) comprises at least one unidirectional recombinase and wherein the recognition sites flanking the nucleic acid sequences are operable with the at least one unidirectional recombinase. In some embodiments, the nucleic acid sequence to be camouflaged is encrypted.

In some aspects, the instant disclosure relates to a method of securely transmitting information to a recipient that is encoded in a nucleic acid sequence comprising providing to a recipient a population of cells comprising a camouflaged nucleic acid sequence, wherein the nucleic acid sequence has been camouflaged using the methods described herein. In some embodiments, the recipient determines the sequence of the camouflaged nucleic acid sequence. In some embodiments, the nucleic acid sequence is encrypted.

### BRIEF DESCRIPTION OF DRAWINGS

FIGS. 1A-1B show samples prepared for NGS sequencing and annotation under blind experimental conditions. FIG. 1A shows Samples 1 and 3: DSD-2α/β and DSD4-α/β/γ/δ were each separately prepared, purified, and mixed at equal concentration in dH2O. Samples 2 and 4: DSD-2α+Cre and DSD4-α+Cre-Flp were prepared under in cognito conditions, purified, and stored in dH2O. DSD-2α/β: 9,549 bp/47.8% GC, Cre: 4,452 bp/49.8% GC, DSD4-α/β/γ/δ: 8,204 bp/46.8% GC, Cre-Flp: 5,769 bp/46.9% GC. FIG. 1B shows samples from FIG. 1A were run on a 1% agarose gel to demonstrate the purity.

FIGS. 2A-2J show DNA camouflage via molecular steganography. FIG. 2A shows a schematic of a 2-state DSD. A 1.6 kb sequence of DNA was placed between inverted loxP sites (triangles). In the presence of Cre under PLtetO-1 regulation, the data in the DSD randomly oscillates between 2 states—α and β. The forward sequencing primer (left arrow) binds directly upstream of the DSD, while the reverse sequencing primer (right arrow) binds 1 kb downstream of the DSD. FIG. 2B shows sequencing of DNA maintained in vivo (DSD-2α+pET28a) or in cognito (DSD-2α+Cre) in E. coli. Samples were taken at 0, 60, and 120 min post Cre induction, plasmids were purified in dH2O and sent for sequencing with the forward sequencing primer. Shown are resulting chromatograms (left) and alignments to DSD-2α (right). FIG. 2C shows Quality Score (QS) and FIG. 2D shows Contiguous Read Length (CRL) scores for sequences in FIG. 2B. FIG. 2E shows distribution of the α and β states of DSD-2 in cognito samples from FIG. 2B. FIG. 2F shows a schematic of a 4-state DSD. Data1 (25 bp), Data2 (1 kb), and Data3 (25 bp) segments were placed between FRT (inner triangles) and loxP (outer triangles) sites. In the presence of a Cre-Flp bicistronic construct under PLtetO-1 regulation, the DSD randomly oscillates between 4 states—α, β, γ, and δ. FIG. 2G shows sequencing data as described above. FIG. 2H shows QS scores as described above. FIG. 2I shows CRL scores as described above. FIG. 2J shows frequency measurements as described above. All experiments were performed in triplicate, error bars represent ±1 standard deviation, and all sequencing reactions and QS/CRL measurements were performed by GENEWIZ Inc. under blind experimental conditions.

FIGS. 3A-3C show that DNA integrity is uncompromised outside of DSD-2α under in cognito conditions. FIG. 3A shows sequencing of DNA maintained in vivo (DSD-2α+pET28a) or in cognito (DSD-2α+Cre) in E. coli. Samples were taken at 0, 60, and 120 min post Cre induction with 100 ng/mL aTc, plasmids were purified and diluted to 60 ng/μL in dH2O and sent for sequencing with the reverse sequencing primer (arrow) that binds 1 kb downstream of DSD-2α. Shown are resulting chromatograms (left) and alignments to DSD-2α (right). FIG. 3B shows Quality Score (QS) and FIG. 3C shows Contiguous Read Length (CRL) scores for sequences in FIG. 3A. All experiments were performed in triplicate, error bars represent ±1 standard deviation, and all sequencing reactions and QS/CRL measurements were performed by GENEWIZ Inc. under blind experimental conditions.

FIGS. 4A-4C show that shuffling of DSD-2αp15A leads to data excision. FIG. 4A shows sequencing of DNA maintained in vivo (DSD-2αp15A+pET28a) or in cognito (DSD-2αp15A+Cre) in E. coli. Samples were taken at 0, 60, and 120 min post Cre induction with 100 ng/mL aTc, plasmids were purified and diluted to 60 ng/μL in dH2O and sent for sequencing with the forward sequencing primer (arrow). Shown are resulting chromatograms (left) and alignments to DSD-2αp15A (right). The DSD was deleted likely due to cross-reaction between loxP sites on different cellular plasmid copies since DSD-2αp15A was on a multiple copy plasmid. FIG. 4B shows Quality Score (QS) and FIG. 4C shows Contiguous Read Length (CRL) scores for sequences in FIG. 4A. All experiments were performed in triplicate, error bars represent ±1 standard deviation, and all sequencing reactions and QS/CRL measurements were performed by GENEWIZ Inc. under blind experimental conditions.

FIGS. 5A-5D show user-control over switching DNA maintained in vivo to in cognito. FIG. 5A shows that to produce a recombinase vector that can be introduced in to cells to shuffle DSDs and then be easily removed, Cre was cloned in to a temperature sensitive plasmid to create Cre^{ts}. DNA maintained in vivo (DSD-2α+pET28a) or in cognito (DSD-2α+Cre^{ts}) in E. coli were cultured overnight at 30° C. and 300 rpm, subsequently Cre was induced with 100 ng/mL aTc and the samples were incubated at 37° C. and 300 rpm for 6 hr. Cell were then diluted 1:10000 and grown overnight at 42° C. and 300 rpm to cure out Cre^{ts}, after which plasmids were purified and diluted to 30 ng/μL in dH2O and sent for sequencing with the forward sequencing primer (arrow). Shown are resulting chromatograms (left) and alignments to DSD-2α (right). FIG. 5B shows Quality Score (QS) and FIG. 5C shows Contiguous Read Length (CRL) scores for sequences in FIG. 5A. FIG. 5D shows DNA isolated from samples in FIG. 5A before (−42° C.) or after (+42° C.) DNA shuffling. 100 ng of purified DNA was run on a 1% agarose gel for 30 min at 130V. All experiments were performed in triplicate, error bars represent ±1 standard deviation, and all sequencing reactions and QS/CRL measurements were performed by GENEWIZ Inc. under blind experimental conditions.

FIG. 6 shows a graphical illustration of DNA camouflage. Conventional DNA encoding includes conversion of digital data files (stored in a computer) into a language that can be written in libraries of DNA molecules, where the data is divided into packets and encoded linearly (for example as shown by SEQ ID NO: 9). Subsequently, the DNA molecules are sequenced and the data is read and converted back to the original formats. With this form of molecular steganography, we introduce a physical security layer (border) where the data packets are shuffled based on a known order to obfuscate interpretation by unauthorized individuals.

FIGS. 7A-7D show recombinase and recombinase-free methods of achieving DNA camouflage. FIG. 7A shows one embodiment of a 2-state device. Top: Schematic of a 2-state DSD with the data packet shuffled by Cre. Bottom: Sequencing of the 2-state DSD data packet in the absence and presence of Cre. FIG. 7B shows one embodiment of a 2-state switchable device. Top: Schematic of a 2-state DSD with the data packet shuffled by Cre^{ts} where Cre is encoded on a temperature sensitive plasmid. Bottom: Sequencing of the 2-state DSD data packet in the absence and presence of Cre^{ts}. FIG. 7C shows one embodiment of a 4-state device. Top: Schematic of a 4-state DSD with three data packets shuffled by Cre and Flp. Bottom: Sequencing of the 4-state DSD data packets in the absence and presence of Cre and Flp. FIG. 7D shows one embodiment of an addiction module. Left: Schematic of the addiction module expression and covalent assembly of SpyTag-Bla and YcbK-SpyCatcher fusion constructs are required to impart Amp resistance (FIG. 11). Right: Efficiency of individual and dual transformation pBZ51 and pBZ52 in E. coli DH5α (control: all plasmids express Kan resistance).

FIGS. 8A-8C show DNA integrity is uncompromised outside of DSD-2α under in cognito maintenance. FIG. 8A shows sequencing of DNA maintained in vivo (DSD-2α+pET28a) and in cognito (DSD-2α+Cre) in E. coli. Samples were taken at 0, 60, and 120 min post Cre induction, plasmids were purified in dH₂O and sent for sequencing with the reverse primer (arrow) that binds 1 kb downstream of DSD-2α. Shown are resulting chromatograms (left) and alignments to DSD-2α (right). FIG. 8B shows Quality Score (QS) and FIG. 8C shows Contiguous Read Length (CRL) scores for sequences in FIG. 8A. All experiments were performed in triplicate, error bars represent ±1 standard deviation, and all sequencing reactions and QS/CRL measurements were performed by GENEWIZ Inc. under blind experimental conditions.

FIGS. 9A-9B show alignment of the NGS identified data sequences to the DNA templates for the DSD-2 samples. FIG. 9A shows DSD-2α. FIG. 9B shows DSD-23.

FIGS. 10A-10D show alignment of the NGS identified data sequences to the DNA templates for the DSD-4 samples. FIG. 10A shows DSD-4α. FIG. 10B shows DSD-43. FIG. 10C shows DSD-4γ. FIG. 10D shows DSD-4δ.

FIG. 11 shows a graphical illustration of programmable isopeptide-based post-translational protein assembly using SpyTag and SpyCatcher. In this embodiment, SpyTag attaches a secretion tag to Bla via Spycatcher, which is linked to the Tat secretion tag YcbK.

FIG. 12 shows agarose gel electrophoresis of a single bacterial colony extraction which yielded both addiction molecule plasmids (e.g., pBZ51 and pBZ52), demonstrating that the two plasmids were stably maintained together.

### DETAILED DESCRIPTION OF INVENTION

In some aspects, the instant disclosure relates to the use of steganographic methods to camouflage information encoded on nucleic acids.

In some embodiments, the instant disclosure relates to a method for camouflaging a nucleic acid sequence comprising: preparing a pool of recombinant nucleic acid constructs, wherein at least one of the constructs comprises the nucleic acid sequence to be camouflaged and wherein the pool is heterogeneous with respect to the orientation of the nucleic acid sequence to be camouflaged. In some embodiments, the pool of constructs is obtained by nucleic acid synthesis. In some embodiments, the pool of constructs is maintained in a cell or cells. In some embodiments, the pool of constructs is maintained in a non-cellular environment.

In some embodiments, the instant disclosure relates to a method for camouflaging a nucleic acid sequence comprising: (a) preparing a recombinant nucleic acid construct that comprises the nucleic acid sequence to be camouflaged, wherein the nucleic acid sequence is flanked by opposing recognition sites of a bidirectional recombinase; (b) introducing the recombinant nucleic acid construct into a cell that comprises the bidirectional recombinase; (c) culturing the cell under conditions in which the cell multiplies to produce a plurality of cells; and, (d) creating a population of cells that is heterogeneous with respect to the orientation of the nucleic acid sequence by culturing the plurality of cells under conditions in which the bidirectional recombinase is expressed, whereby the nucleic acid sequence flanked by the opposing recognition sites of the bidirectional recombinase is inverted in a portion of the plurality of cells, thereby camouflaging the nucleic acid sequence.

In some embodiments, the instant disclosure relates to a method for camouflaging a nucleic acid sequence comprising: (a) preparing a recombinant nucleic acid construct that comprises the nucleic acid sequence to be camouflaged, wherein the nucleic acid sequence is flanked by opposing recognition sites of a bidirectional recombinase; (b) combining a plurality of the construct of (a) and a functional bidirectional recombinase in vitro, whereby the nucleic acid sequence flanked by the opposing recognition sites of the bidirectional recombinase is inverted, thereby producing a heterogeneous population of camouflaged constructs; and, (c) maintaining the heterogeneous population of (b) in a non-cellular environment.

In some embodiments, the nucleic acid comprises a protein-encoding gene. In some embodiments, the nucleic acid sequence is dispersed across a plurality of nucleic acids; each of the one or more nucleic acids is comprised of a plurality of segments. In some embodiments, at least two of the plurality of nucleic acids or the plurality of segments are flanked by opposing recognition sites of at least two different bidirectional recombinases, and wherein the cell or plurality of cells comprises the at least two different bidirectional recombinases.

As used herein “nucleic acid” refers to a DNA or RNA molecule. Nucleic acids are polymeric macromolecules comprising a plurality of nucleotides. In some embodiments, the nucleotides are deoxyribonucleotides or ribonucleotides. In some embodiments, the nucleotides comprising the nucleic acid are selected from the group consisting of adenine, guanine, cytosine, thymine, uracil and inosine. In some embodiments, the nucleotides comprising the nucleic acid are modified nucleotides. Methods of modifying nucleotides are generally known in the art. Non-limiting examples of nucleotide modifications include phosphorothioate backbone modifications, 2′-O-methyl group sugar modifications and the substitution of non-naturally occurring nucleotide bases (for example, nucleotides derivatized at the 5-, 6-, 7- or 8-position). In some embodiments, the nucleic acids of the instant disclosure are synthetic. The term “synthetic” refers to a nucleic acid molecule that is constructed via the joining nucleotides by a synthetic or non-natural method. One non-limiting example of a synthetic method is solid-phase oligonucleotide synthesis. In some embodiments, the nucleic acids of the instant disclosure are isolated. In some embodiments, the nucleic acids of the instant disclosure are selected from the group consisting of synthetic DNA, linear DNA and genomic DNA.

In some embodiments, the instant disclosure relates to recombinant nucleic acid constructs. The term “recombinant construct” refers to an artificially constructed molecule comprising a nucleic acid (e.g., DNA) insert and a vector capable of artificially carrying foreign genetic material into another cell. In some embodiments, vectors carry common functional elements including an origin of replication, a multicloning site and a selectable marker. In some embodiments, the selectable marker is a bacterial resistance gene, for example β-lactamase, Neo or mFabI. Non-limiting examples of vectors include plasmids, viral vectors, cosmids, and artificial chromosomes. In some embodiments, the vector is a high-copy plasmid. In some embodiments, the vector is a low-copy plasmid. In some embodiments, the recombinant constructs of the instant disclosure are maintained inside cells. In some embodiments, the recombinant constructs of the instant disclosure are maintained in a non-cellular environment.

In some embodiments, the recombinant nucleic acid constructs comprising the nucleic acid sequence to be camouflaged are contained in one or more host cells. Host cells are living cells that permit the existence and replication of recombinant nucleic acid constructs. In some embodiments of the method, the cell is a prokaryotic cell. In some embodiments of the method, the cell is a eukaryotic cell. In some embodiments, a recombinant construct may be integrated into the genome of the cell. In some embodiments, the recombinant nucleic acid construct is integrated into the genomes of the plurality of cells.

In some aspects, the instant disclosure relates to the surprising discovery that genetic recombination is useful for camouflaging information encoded on nucleic acids. Genetic recombination is an enzyme-driven biological process by which nucleotide sequences are exchanged between two DNA molecules or exchanged within the same DNA molecule. In some embodiments, the genetic recombination described herein takes place between two DNA molecules. In some embodiments, the two DNA molecules are similar or identical and the genetic recombination is homologous recombination. In some embodiments, the genetic recombination described herein takes place within the same DNA molecule, for example the inversion of a sequence fragment within the same gene. In some embodiments, the DNA rearrangement takes place between segments possessing only a limited degree of sequence homology and is site-specific recombination. Recombinases are enzymes that mediate site-specific recombination by binding to nucleic acids via conserved recognition sites and mediating at least one of the following forms of DNA rearrangement: integration, excision/resolution and/or inversion. Recombinases are generally classified into two families of proteins, tyrosine recombinases (YR) and serine recombinases (SR). However, recombinases may also be classified according to their directionality (i.e., bidirectional or unidirectional). Bidirectional recombinases bind to identical recognition sites and therefore mediate reversible recombination. Non-limiting examples of identical recognition sites for bidirectional recombinases include loxP, FRT and RS recognition sites. Unidirectional recombinases bind to non-identical recognition sites and therefore mediate irreversible recombination. In some embodiments, the methods described herein utilize unidirectional recombinases to camouflage nucleic acid sequences.

In some embodiments, the methods described herein utilize bidirectional recombinases to camouflage nucleic acid sequences. Examples of bidirectional recombinases include, but are not limited to, Cre, FLP, R, IntA, Tn3 resolvase, Hin invertase and Gin invertase. In some embodiments of the method described herein, the cell(s) comprising recombinant nucleic acid constructs further comprise at least one of Cre, Flp and R bidirectional recombinases, wherein the recognition sites flanking the nucleic acid sequence are operable with the at least one bidirectional recombinases and are selected from loxP, FRT and RS recognition sites. In some embodiments, the cell(s) comprise two or more bidirectional recombinases, wherein the recognition sites flanking the nucleic acid are operable with the two or more bidirectional recombinases. In some embodiments, the cell(s) comprises at least one unidirectional recombinase and wherein the recognition sites flanking the nucleic acid sequences are operable with the at least one unidirectional recombinase.

Methods of expressing bidirectional recombinases in a cell are also disclosed herein. In some embodiments, the recombinant nucleic acid construct comprising the bidirectional recombinase may be transiently expressed in the cell. In some embodiments, the recombinant nucleic acid construct comprising the bidirectional recombinase may be stably expressed in the cell. The level of protein expression of the bidirectional recombinase within the cell may also be adjusted. In some embodiments, the bidirectional recombinase is constitutively expressed in the cell. As used herein, the term “constitutively expressed” refers to the constant transcription and translation of a gene across all known conditions. In some embodiments of the method, the bidirectional recombinase(s) is/are expressed constitutively in the plurality of cells. In some embodiments, the expression of the bidirectional recombinase is induced. Inducible expression refers to the expression of a gene only when a particular substance or condition is present in the cellular environment. Inducible expression methods are well-known in the art, for example conditional promoters such as tetracycline controlled transcriptional activation (Tet-on and Tet-off), thermoinducible expression systems and temperature sensitive plasmids. In some embodiments of the method, the bidirectional recombinase(s) is/are expressed in the cell on one or more temperature sensitive plasmids. In some embodiments, the bidirectional recombinase(s) is/are expressed under control of one or more conditional promoters in the cell or the plurality of cells.

In some aspects, the disclosure relates to the maintenance of a population of cells that is heterogeneous with respect to the orientation of a nucleic acid sequence to be camouflaged. In some embodiments, the population of cells comprises more than one (e.g., 2, 3, 4, 5 or more) nucleic acid sequences to be camouflaged. Nucleic acid sequences to be camouflaged can be located on the same plasmid or on different plasmids. However, without wishing to be bound by any particular theory, two plasmids with the same origin of replication and selection marker cannot be stably maintained within a single cell over extended periods of time.

The disclosure is based, in part, on the discovery that addiction molecules can be used to stably maintain two plasmids having the same origin of replication in a single cell. As used herein, “addiction molecule” refers to a molecule essential for bacterial survival that is produced by covalent assembly from two separate plasmids having the same origin of replication. For example, a bacterial selection marker (e.g., the Bla gene, responsible for ampicillin resistance) can be split into two fragments and fused to a dimerizing molecule (e.g., the SpyTag/SpyCatcher system, as described by Zakeri et al. Proc Natl Acad Sci USA. 2012 Mar. 20; 109(12):E690-7.); each fragment is then inserted into a plasmid having the same origin of replication. Expression of each fragment in bacteria results in reconstitution of the functional Bla gene and resistance to ampicillin selection. In some embodiments, a nucleic acid construct as described by the disclosure comprises one or more “addiction molecules”.

In some embodiments, the nucleic acid sequence to be camouflaged is encrypted. The information contained within the camouflaged nucleic acid sequence may be further protected by encryption. Encryption of information into nucleic acids may be achieved by translating information into nucleic acid message sequence using a cryptographic key and synthesizing nucleic acid molecules comprising fragments of the nucleic acid message sequence interspersed with highly variable randomized nucleic acid sequence. In some embodiments, the nucleic acid to be camouflaged may be encrypted according to the methods disclosed in the United States provisional application filed of even date (Ser. No. 62/069,994, filed under Attorney Docket No. M0656.70337US00) and incorporated by reference in its entirety herein. In some embodiments, the camouflaged encrypted nucleic acid is maintained in a non-cellular environment.

In some aspects, the instant disclosure relates to a method of securely transmitting information to a recipient that is encoded in a nucleic acid sequence comprising providing to a recipient a population of cells comprising a camouflaged nucleic acid sequence, wherein the nucleic acid sequence has been camouflaged using the methods described herein. In some embodiments, the recipient determines the sequence of the camouflaged nucleic acid sequence. In some embodiments, the nucleic acid sequence is encrypted as described in the United States provisional application filed of even date (Ser. No. 62/069,994, filed under Attorney Docket No. M0656.70337US00).

### Examples

### Example 1: Materials and Methods

### Plasmids

Standard molecular biology techniques were used to clones all constructs. KOD Hot Start DNA Polymerase (VWR) was used for all PCR reactions as per manufacturer recommended conditions, and all primers were produced by IDT. PCR reactions were performed in an Eppendorf Mastercycler Gradient. Gibson Assembly Master Mix (NEB) was used for all plasmid assembly reactions of DNA fragments as per manufacturer recommended conditions, where 25 bp homology arms were used for DNA fragments assembly. Assembled constructs were transformed in to E. coli DH5αPRO (F⁻ φ80lacZΔM15 Δ(lacZYA-argF)U169 deoR recA1 endA1 hsdR17(rk⁻, mk⁺) phoA supE44 thi-1 gyrA96 relA1 λ⁻, PN25/tet^{R}, Placiq/lacI, Sp^{r}). Cultures were grown in LB supplemented with either chloramphenicol (12.5 μg/mL) or kanamycin (50 μg/mL) as required. Individual parts were either amplified from varying sources or introduced synthetically with primers and subsequently assembled. Detailed information for all plasmids in this study are outlined in Table 1. Plasmids were constructed using Gibson Assembly by assembling natural genetic and synthetic parts. All constructs were sequence verified at GENEWIZ Inc (Cambridge, Mass.).

TABLE 1
Identity, plasmid, and sequence information of constructs used.

| | Plas- | | | | SEQ |
|---|---|---|---|---|---|
| Con- | mid | Plasmid | | | ID |
| struct | Name | Backbone | Sequence | Legend | NO: |
| Cre | pBZ14 | pET28a | TCCCTATCAGTGATAGAGATTGACATCCCTATCAGTGATAGA | P_{LtetO-1} | 1 |
| | | (origin | GATACTGAGCACATCAGCAGGACGCACTGACCACTTTAAGA | Cre | |
| | | and | AGGAGATATACCATGGCCAATTTACTGACCGTACACCAAAAT | Terminator | |
| | | Kan^{R} | TTGCCTGCATTACCGGTCGATGCAACGAGTGATGAGGTTCG | Spacer | |
| | | only) | CAAGAACCTGATGGACATGTTCAGGGATCGCCAGGCGTTTT | | |
| | | | CTGAGCATACCTGGAAAATGCTTCTGTCCGTTTGCCGGTCG | | |
| | | | TGGGCGGCATGGTGCAAGTTGAATAACCGGAAATGGTTTCC | | |
| | | | CGCAGAACCTGAAGATGTTCGCGATTATCTTCTATATCTTCA | | |
| | | | GGCGCGCGGTCTGGCAGTAAAAACTATCCAGCAACATTTGG | | |
| | | | GCCAGCTAAACATGCTTCATCGTCGGTCCGGGCTGCCACG | | |
| | | | ACCAAGTGACAGCAATGCTGTTTCACTGGTTATGCGGCGGA | | |
| | | | TCCGAAAAGAAAACGTTGATGCCGGTGAACGTGCAAAACAG | | |
| | | | GCTCTAGCGTTCGAACGCACTGATTTCGACCAGGTTCGTTC | | |
| | | | ACTCATGGAAAATAGCGATCGCTGCCAGGATATACGTAATC | | |
| | | | TGGCATTTCTGGGGATTGCTTATAACACCCTGTTACGTATAG | | |
| | | | CCGAAATTGCCAGGATCAGGGTTAAAGATATCTCACGTACT | | |
| | | | GACGGTGGGAGAATGTTAATCCATATTGGCAGAACGAAAAC | | |
| | | | GCTGGTTAGCACCGCAGGTGTAGAGAAGGCACTTAGCCTG | | |
| | | | GGGGTAACTAAACTGGTCGAGCGATGGATTTCCGTCTCTGG | | |
| | | | TGTAGCTGATGATCCGAATAACTACCTGTTTTGCCGGGTCA | | |
| | | | GAAAAAATGGTGTTGCCGCGCCATCTGCCACCAGCCAGCTA | | |
| | | | TCAACTCGCGCCCTGGAAGGGATTTTTGAAGCAACTCATCG | | |
| | | | ATTGATTTACGGCGCTAAGGATGACTCTGGTCAGAGATACC | | |
| | | | TGGCCTGGTCTGGACACAGTGCCCGTGTCGGAGCCGCGCG | | |
| | | | AGATATGGCCCGCGCTGGAGTTTCAATACCGGAGATCATGC | | |
| | | | AAGCTGGTGGCTGGACCAATGTAAATATTGTCATGAACTATA | | |
| | | | TCCGTAACCTGGATAGTGAAACAGGGGCAATGGTGCGCCT | | |
| | | | GCTGGAAGATGGCGATTAAGTCGACAACCTAGGAAAAACCT | | |
| | | | GAGGAAAATGCATAGCTAGAGGCATCAAATAAAACGAAAGG | | |
| | | | CTCAGTCGAAAGACTGGGCCTTTCGTTTTATCTGTTGTTTGT | | |
| | | | CGGTGAACGCTCTCCTGAGTAGGACAAATCCGCCGGGAGC | | |
| | | | GGATTTGAACGCTGCGAAGCAACGGCCCGGAGGGTGGCG | | |
| | | | GGCAGGACGCCCGCCATAAACTGCCAGGCATCAAATTAAG | | |
| | | | CAGAAGGCCATCCTGACGGATGGCCTTTTTGCGTTTCTACA | | |
| | | | AA | | |
| Cre^{ts} | pBZ20 | pKD46 | TCCCTATCAGTGATAGAGATTGACATCCCTATCAGTGATAGA | P_{LtetO-1} | 2 |
| | | (origin | GATACTGAGCACATCAGCAGGACGCACTGACCACTTTAAGA | Cre | |
| | | and | AGGAGATATACCATGGCCAATTTACTGACCGTACACCAAAAT | Terminator | |
| | | Amp^{R} | TTGCCTGCATTACCGGTCGATGCAACGAGTGATGAGGTTCG | Spacer | |
| | | only) | CAAGAACCTGATGGACATGTTCAGGGATCGCCAGGCGTTTT | | |
| | | | CTGAGCATACCTGGAAAATGCTTCTGTCCGTTTGCCGGTCG | | |
| | | | TGGGCGGCATGGTGCAAGTTGAATAACCGGAAATGGTTTCC | | |
| | | | CGCAGAACCTGAAGATGTTCGCGATTATCTTCTATATCTTCA | | |
| | | | GGCGCGCGGTCTGGCAGTAAAAACTATCCAGCAACATTTGG | | |
| | | | GCCAGCTAAACATGCTTCATCGTCGGTCCGGGCTGCCACG | | |
| | | | ACCAAGTGACAGCAATGCTGTTTCACTGGTTATGCGGCGGA | | |
| | | | TCCGAAAAGAAAACGTTGATGCCGGTGAACGTGCAAAACAG | | |
| | | | GCTCTAGCGTTCGAACGCACTGATTTCGACCAGGTTCGTTC | | |
| | | | ACTCATGGAAAATAGCGATCGCTGCCAGGATATACGTAATC | | |
| | | | TGGCATTTCTGGGGATTGCTTATAACACCCTGTTACGTATAG | | |
| | | | CCGAAATTGCCAGGATCAGGGTTAAAGATATCTCACGTACT | | |
| | | | GACGGTGGGAGAATGTTAATCCATATTGGCAGAACGAAAAC | | |
| | | | GCTGGTTAGCACCGCAGGTGTAGAGAAGGCACTTAGCCTG | | |
| | | | GGGGTAACTAAACTGGTCGAGCGATGGATTTCCGTCTCTGG | | |
| | | | TGTAGCTGATGATCCGAATAACTACCTGTTTTGCCGGGTCA | | |
| | | | GAAAAAATGGTGTTGCCGCGCCATCTGCCACCAGCCAGCTA | | |
| | | | TCAACTCGCGCCCTGGAAGGGATTTTTGAAGCAACTCATCG | | |
| | | | ATTGATTTACGGCGCTAAGGATGACTCTGGTCAGAGATACC | | |
| | | | TGGCCTGGTCTGGACACAGTGCCCGTGTCGGAGCCGCGCG | | |
| | | | AGATATGGCCCGCGCTGGAGTTTCAATACCGGAGATCATGC | | |
| | | | AAGCTGGTGGCTGGACCAATGTAAATATTGTCATGAACTATA | | |
| | | | TCCGTAACCTGGATAGTGAAACAGGGGCAATGGTGCGCCT | | |
| | | | GCTGGAAGATGGCGATTAAGTCGACAACCTAGGAAAAACCT | | |
| | | | GAGGAAAATGCATAGCTAGAGGCATCAAATAAAACGAAAGG | | |
| | | | CTCAGTCGAAAGACTGGGCCTTTCGTTTTATCTGTTGTTTGT | | |
| | | | CGGTGAACGCTCTCCTGAGTAGGACAAATCCGCCGGGAGC | | |
| | | | GGATTTGAACGCTGCGAAGCAACGGCCCGGAGGGTGGCG | | |
| | | | GGCAGGACGCCCGCCATAAACTGCCAGGCATCAAATTAAG | | |
| | | | CAGAAGGCCATCCTGACGGATGGCCTTTTTGCGTTTCTACA | | |
| | | | AA | | |
| Cre-Flp | pBZ17 | pET28a | TCCCTATCAGTGATAGAGATTGACATCCCTATCAGTGATAGA | P_{LtetO-1} | 3 |
| | | (origin | GATACTGAGCACATCAGCAGGACGCACTGACCACTTTAAGA | Cre | |
| | | and | AGGAGATATACCATGGCCAATTTACTGACCGTACACCAAAAT | Flp | |
| | | Kan^{R} | TTGCCTGCATTACCGGTCGATGCAACGAGTGATGAGGTTCG | Terminator | |
| | | only) | CAAGAACCTGATGGACATGTTCAGGGATCGCCAGGCGTTTT | Spacer | |
| | | | CTGAGCATACCTGGAAAATGCTTCTGTCCGTTTGCCGGTCG | | |
| | | | TGGGCGGCATGGTGCAAGTTGAATAACCGGAAATGGTTTCC | | |
| | | | CGCAGAACCTGAAGATGTTCGCGATTATCTTCTATATCTTCA | | |
| | | | GGCGCGCGGTCTGGCAGTAAAAACTATCCAGCAACATTTGG | | |
| | | | GCCAGCTAAACATGCTTCATCGTCGGTCCGGGCTGCCACG | | |
| | | | ACCAAGTGACAGCAATGCTGTTTCACTGGTTATGCGGCGGA | | |
| | | | TCCGAAAAGAAAACGTTGATGCCGGTGAACGTGCAAAACAG | | |
| | | | GCTCTAGCGTTCGAACGCACTGATTTCGACCAGGTTCGTTC | | |
| | | | ACTCATGGAAAATAGCGATCGCTGCCAGGATATACGTAATC | | |
| | | | TGGCATTTCTGGGGATTGCTTATAACACCCTGTTACGTATAG | | |
| | | | CCGAAATTGCCAGGATCAGGGTTAAAGATATCTCACGTACT | | |
| | | | GACGGTGGGAGAATGTTAATCCATATTGGCAGAACGAAAAC | | |
| | | | GCTGGTTAGCACCGCAGGTGTAGAGAAGGCACTTAGCCTG | | |
| | | | GGGGTAACTAAACTGGTCGAGCGATGGATTTCCGTCTCTGG | | |
| | | | TGTAGCTGATGATCCGAATAACTACCTGTTTTGCCGGGTCA | | |
| | | | GAAAAAATGGTGTTGCCGCGCCATCTGCCACCAGCCAGCTA | | |
| | | | TCAACTCGCGCCCTGGAAGGGATTTTTGAAGCAACTCATCG | | |
| | | | ATTGATTTACGGCGCTAAGGATGACTCTGGTCAGAGATACC | | |
| | | | TGGCCTGGTCTGGACACAGTGCCCGTGTCGGAGCCGCGCG | | |
| | | | AGATATGGCCCGCGCTGGAGTTTCAATACCGGAGATCATGC | | |
| | | | AAGCTGGTGGCTGGACCAATGTAAATATTGTCATGAACTATA | | |
| | | | TCCGTAACCTGGATAGTGAAACAGGGGCAATGGTGCGCCT | | |
| | | | GCTGGAAGATGGCGATTAATAAAAGCTTCTCTAGAAATAATT | | |
| | | | TTGTTTAACTTTAAGAAGGAGATATACCATGAGCCAATTTGA | | |
| | | | TATATTATGTAAAACACCACCTAAGGTCCTGGTTCGTCAGT | | |
| | | | TTGTGGAAAGGTTTGAAAGACCTTCAGGGGAAAAAATAGC | | |
| | | | ATCATGTGCTGCTGAACTAACCTATTTATGTTGGATGATTA | | |
| | | | CTCATAACGGAACAGCAATCAAGAGAGCCACATTCATGAG | | |
| | | | CTATAATACTATCATAAGCAATTCGCTGAGTTTCGATATTG | | |
| | | | TCAACAAATCACTCCAGTTTAAATACAAGACGCAAAAAGC | | |
| | | | AACAATTCTGGAAGCCTCATTAAAGAAATTAATTCCTGCTT | | |
| | | | GGGAATTTACAATTATTCCTTACAATGGACAAAAACATCAA | | |
| | | | TCTGATATCACTGATATTGTAAGTAGTTTGCAATTACAGTTC | | |
| | | | GAATCATCGGAAGAAGCAGATAAGGGAAATAGCCACAGT | | |
| | | | AAAAAAATGCTTAAAGCACTTCTAAGTGAGGGTGAAAGCA | | |
| | | | TCTGGGAGATCACTGAGAAAATACTAAATTCGTTTGAGTAT | | |
| | | | ACCTCGAGATTTACAAAAACAAAAACTTTATACCAATTCCT | | |
| | | | CTTCCTAGCTACTTTCATCAATTGTGGAAGATTCAGCGATA | | |
| | | | TTAAGAACGTTGATCCGAAATCATTTAAATTAGTCCAAAAT | | |
| | | | AAGTATCTGGGAGTAATAATCCAGTGTTTAGTGACAGAGA | | |
| | | | CAAAGACAAGCGTTAGTAGGCACATATACTTCTTTAGCGC | | |
| | | | AAGGGGTAGGATCGATCCACTTGTATATTTGGATGAATTTT | | |
| | | | TGAGGAACTCTGAACCAGTCCTAAAACGAGTAAATAGGAC | | |
| | | | CGGCAATTCTTCAAGCAACAAACAGGAATACCAATTATTA | | |
| | | | AAAGATAACTTAGTCAGATCGTACAACAAGGCTTTGAAGA | | |
| | | | AAAATGCGCCTTATCCAATCTTTGCTATAAAGAATGGCCCA | | |
| | | | AAATCTCACATTGGAAGACATTTGATGACCTCATTTCTGTC | | |
| | | | AATGAAGGGCCTAACGGAGTTGACTAATGTTGTGGGAAAT | | |
| | | | TGGAGCGATAAGCGTGCTTCTGCCGTGGCCAGGACAACGT | | |
| | | | ATACTCATCAGATAACAGCAATACCTGATCACTACTTCGCA | | |
| | | | CTAGTTTCTCGGTACTATGCATATGATCCAATATCAAAGGA | | |
| | | | AATGATAGCATTGAAGGATGAGACTAATCCAATTGAGGAG | | |
| | | | TGGCAGCATATAGAACAGCTAAAGGGTAGTGCTGAAGGAA | | |
| | | | GCATACGATACCCCGCATGGAATGGGATAATATCACAGGA | | |
| | | | GGTACTAGACTACCTTTCATCCTACATAAATTAATAAGTCG | | |
| | | | ACAACCTAGGAAAAACCTGAGGAAAATGCATAGCTAGAGGC | | |
| | | | ATCAAATAAAACGAAAGGCTCAGTCGAAAGACTGGGCCTTT | | |
| | | | CGTTTTATCTGTTGTTTGTCGGTGAACGCTCTCCTGAGTAGG | | |
| | | | ACAAATCCGCCGGGAGCGGATTTGAACGCTGCGAAGCAAC | | |
| | | | GGCCCGGAGGGTGGCGGGCAGGACGCCCGCCATAAACTG | | |
| | | | CCAGGCATCAAATTAAGCAGAAGGCCATCCTGACGGATGGC | | |
| | | | CTTTTTGCGTTTCTACAAA | | |
| DSD-2α | pBZ22 | pBAC-LacZ | ATAACTTCGTATAATGTATGCTATACGAAGTTATGCAGTTTC | loxP | 4 |
| | | (F′/oriV | ATTTGATGCTCGATGAGTTTTTCTAAGAATTAATTCATGAGC | Data | |
| | | origins | GGATACATATTTGAATGTATTTAGAAAAATAAACAAATAGGG | | |
| | | and | GTTCCGCGCACATTTCCCCGAAAAGTGCCACCTAGGTATCT | | |
| | | Cam^{R}) | GGCACTACGTTCAGGTAACCTGAAGCTCGAATCCAGTACTC | | |
| | | | GACGTCTCTAGGGCGGCGGATTTGTCCTACTCAGGAGAGC | | |
| | | | GTTCACCGACAAACAACAGATAAAACGAAAGGCCCAGTCTT | | |
| | | | TCGACTGAGCCTTTCGTTTTATTTGATGCCTCTAGCACGCGT | | |
| | | | ACCTGGTGGCGCGCCTTATTTGTATAGTTCATCCATGCCAT | | |
| | | | GTGTAATCCCAGCAGCTGTTACAAACTCAAGAAGGACCATG | | |
| | | | TGGTCTCTCTTTTCGTTGGGATCTTTCGAAAGGGCAGATTGT | | |
| | | | GTGGACAGGTAATGGTTGTCTGGTAAAAGGACAGGGCCATC | | |
| | | | GCCAATTGGAGTATTTTGTTGATAATGGTCTGCTAGTTGAAC | | |
| | | | GCTTCCATCTTCAATGTTGTGTCTAATTTTGAAGTTAACTTTG | | |
| | | | ATTCCATTCTTTTGTTTGTCTGCCATGATGTATACATTGTGTG | | |
| | | | AGTTATAGTTGTATTCCAATTTGTGTCCAAGAATGTTTCCATC | | |
| | | | TTCTTTAAAATCAATACCTTTTAACTCGATTCTATTAACAAGG | | |
| | | | GTATCACCTTCAAACTTGACTTCAGCACGTGTCTTGTAGTTC | | |
| | | | CCGTCATCTTTGAAAAATATAGTTCTTTCCTGTACATAACCTT | | |
| | | | CGGGCATGGCACTCTTGAAAAAGTCATGCTGTTTCATATGAT | | |
| | | | CTGGGTATCTCGCAAAGCATTGAACACCATAACCGAAAGTA | | |
| | | | GTGACAAGTGTTGGCCATGGAACAGGTAGTTTTCCAGTAGT | | |
| | | | GCAAATAAATTTAAGGGTAAGTTTTCCGTATGTTGCATCACC | | |
| | | | TTCACCCTCTCCACTGACAGAAAATTTGTGCCCATTAACATC | | |
| | | | ACCATCTAATTCAACAAGAATTGGGACAACTCCAGTGAAAAG | | |
| | | | TTCTTCTCCTTTACGCATGGTATATCTCCTTCTTAAAGTGGT | | |
| | | | CAGTGCGTCCTGCTGATGTGCTCAGTATCTTGTTATCCGCT | | |
| | | | CACAATGTAAATTGTTATCCGCTCACAATTGTATCCGCTCAT | | |
| | | | GAATTAATTCTTAGGCATATTCAAATCGTTTTCGTTACCGCTT | | |
| | | | GCAGGCATCATGACAGAACACTACTTCCTATAAACGCTACA | | |
| | | | CAGGCTCCTGAGATTAATAATGCGGATCTGTCCCAGACTAA | | |
| | | | TAATCAGACCGACGAAGAAACCAATTGTCCATATTGCATCAG | | |
| | | | ACATTGCCGTCACTGCGTCTTTTACTGGCTCTTCTCGCTAAC | | |
| | | | CAAACCGGTAACCCCGCTTATTAAAAGCATTCTGTAACAAAG | | |
| | | | CGGGACCAAAGCCATGACAAAAACGCGTAACAAAAGTGTCT | | |
| | | | ATAATCACGGCAGAAAAGTCCACATTGATTATTTGCACGGC | | |
| | | | GTCACACTTTGCTATGCCATAGCATTTTTATCCATAAGATTA | | |
| | | | GCGGATCCTACCTGACGCTTTTTATCGCAACTCTCTACTGTT | | |
| | | | TCTCCATAGCTAGCATAACTTCGTATAGCATACATTATACGA | | |
| | | | AGTTAT | | |
| DSD- | pBZ19 | p15A | ATAACTTCGTATAATGTATGCTATACGAAGTTATGCAGTTTC | loxP | 5 |
| 2α^{p15A} | | (origin | ATTTGATGCTCGATGAGTTTTTCTAAGAATTAATTCATGAGC | Data | |
| | | and Cam^{R} | GGATACATATTTGAATGTATTTAGAAAAATAAACAAATAGGG | | |
| | | only) | GTTCCGCGCACATTTCCCCGAAAAGTGCCACCTAGGTATCT | | |
| | | | GGCACTACGTTCAGGTAACCTGAAGCTCGAATCCAGTACTC | | |
| | | | GACGTCTCTAGGGCGGCGGATTTGTCCTACTCAGGAGAGC | | |
| | | | GTTCACCGACAAACAACAGATAAAACGAAAGGCCCAGTCTT | | |
| | | | TCGACTGAGCCTTTCGTTTTATTTGATGCCTCTAGCACGCGT | | |
| | | | ACCTGGTGGCGCGCCTTATTTGTATAGTTCATCCATGCCAT | | |
| | | | GTGTAATCCCAGCAGCTGTTACAAACTCAAGAAGGACCATG | | |
| | | | TGGTCTCTCTTTTCGTTGGGATCTTTCGAAAGGGCAGATTGT | | |
| | | | GTGGACAGGTAATGGTTGTCTGGTAAAAGGACAGGGCCATC | | |
| | | | GCCAATTGGAGTATTTTGTTGATAATGGTCTGCTAGTTGAAC | | |
| | | | GCTTCCATCTTCAATGTTGTGTCTAATTTTGAAGTTAACTTTG | | |
| | | | ATTCCATTCTTTTGTTTGTCTGCCATGATGTATACATTGTGTG | | |
| | | | AGTTATAGTTGTATTCCAATTTGTGTCCAAGAATGTTTCCATC | | |
| | | | TTCTTTAAAATCAATACCTTTTAACTCGATTCTATTAACAAGG | | |
| | | | GTATCACCTTCAAACTTGACTTCAGCACGTGTCTTGTAGTTC | | |
| | | | CCGTCATCTTTGAAAAATATAGTTCTTTCCTGTACATAACCTT | | |
| | | | CGGGCATGGCACTCTTGAAAAAGTCATGCTGTTTCATATGAT | | |
| | | | CTGGGTATCTCGCAAAGCATTGAACACCATAACCGAAAGTA | | |
| | | | GTGACAAGTGTTGGCCATGGAACAGGTAGTTTTCCAGTAGT | | |
| | | | GCAAATAAATTTAAGGGTAAGTTTTCCGTATGTTGCATCACC | | |
| | | | TTCACCCTCTCCACTGACAGAAAATTTGTGCCCATTAACATC | | |
| | | | ACCATCTAATTCAACAAGAATTGGGACAACTCCAGTGAAAAG | | |
| | | | TTCTTCTCCTTTACGCATGGTATATCTCCTTCTTAAAGTGGT | | |
| | | | CAGTGCGTCCTGCTGATGTGCTCAGTATCTTGTTATCCGCT | | |
| | | | CACAATGTAAATTGTTATCCGCTCACAATTGTATCCGCTCAT | | |
| | | | GAATTAATTCTTAGGCATATTCAAATCGTTTTCGTTACCGCTT | | |
| | | | GCAGGCATCATGACAGAACACTACTTCCTATAAACGCTACA | | |
| | | | CAGGCTCCTGAGATTAATAATGCGGATCTGTCCCAGACTAA | | |
| | | | TAATCAGACCGACGAAGAAACCAATTGTCCATATTGCATCAG | | |
| | | | ACATTGCCGTCACTGCGTCTTTTACTGGCTCTTCTCGCTAAC | | |
| | | | CAAACCGGTAACCCCGCTTATTAAAAGCATTCTGTAACAAAG | | |
| | | | CGGGACCAAAGCCATGACAAAAACGCGTAACAAAAGTGTCT | | |
| | | | ATAATCACGGCAGAAAAGTCCACATTGATTATTTGCACGGC | | |
| | | | GTCACACTTTGCTATGCCATAGCATTTTTATCCATAAGATTA | | |
| | | | GCGGATCCTACCTGACGCTTTTTATCGCAACTCTCTACTGTT | | |
| | | | TCTCCATAGCTAGCATAACTTCGTATAGCATACATTATACGA | | |
| | | | AGTTAT | | |
| DSD-4α | pBZ23 | pBAC-LacZ | ATAACTTCGTATAATGTATGCTATACGAAGTTATGCAGTTTC | loxP | 6 |
| | | (F′/oriV | ATTTGATGCTCGATGAGGAAGTTCCTATTCTCTAGAAAGTA | FRT | |
| | | origins | TAGGAACTTCAAGCTCGAATCCAGTACTCGACGTCTCTAGG | Data1 | |
| | | and | GCGGCGGATTTGTCCTACTCAGGAGAGCGTTCACCGACAAA | Data2 | |
| | | Cam^{R}) | CAACAGATAAAACGAAAGGCCCAGTCTTTCGACTGAGCCTT | Data3 | |
| | | | TCGTTTTATTTGATGCCTCTAGCACGCGTACCTGGTGGCGC | | |
| | | | GCCTTATTTGTATAGTTCATCCATGCCATGTGTAATCCCAGC | | |
| | | | AGCTGTTACAAACTCAAGAAGGACCATGTGGTCTCTCTTTTC | | |
| | | | GTTGGGATCTTTCGAAAGGGCAGATTGTGTGGACAGGTAAT | | |
| | | | GGTTGTCTGGTAAAAGGACAGGGCCATCGCCAATTGGAGTA | | |
| | | | TTTTGTTGATAATGGTCTGCTAGTTGAACGCTTCCATCTTCA | | |
| | | | ATGTTGTGTCTAATTTTGAAGTTAACTTTGATTCCATTCTTTT | | |
| | | | GTTTGTCTGCCATGATGTATACATTGTGTGAGTTATAGTTGT | | |
| | | | ATTCCAATTTGTGTCCAAGAATGTTTCCATCTTCTTTAAAATC | | |
| | | | AATACCTTTTAACTCGATTCTATTAACAAGGGTATCACCTTCA | | |
| | | | AACTTGACTTCAGCACGTGTCTTGTAGTTCCCGTCATCTTTG | | |
| | | | AAAAATATAGTTCTTTCCTGTACATAACCTTCGGGCATGGCA | | |
| | | | CTCTTGAAAAAGTCATGCTGTTTCATATGATCTGGGTATCTC | | |
| | | | GCAAAGCATTGAACACCATAACCGAAAGTAGTGACAAGTGT | | |
| | | | TGGCCATGGAACAGGTAGTTTTCCAGTAGTGCAAATAAATTT | | |
| | | | AAGGGTAAGTTTTCCGTATGTTGCATCACCTTCACCCTCTCC | | |
| | | | ACTGACAGAAAATTTGTGCCCATTAACATCACCATCTAATTC | | |
| | | | AACAAGAATTGGGACAACTCCAGTGAAAAGTTCTTCTCCTTT | | |
| | | | ACGCATGGTATATCTCCTTCTTAAAGTGGTCAGTGCGTCCT | | |
| | | | GCTGATGTGCTCAGTATCTTGTTATCCGCTCACAATGTAAAT | | |
| | | | TGTTATCCGCTCACAATTGTATCCGCTCATGAATTAATTCTTA | | |
| | | | GAAGTTCCTATACTTTCTAGAGAATAGGAACTTCAGGCATT | | |
| | | | GATGGAATCGTAGTCTCAATAACTTCGTATAGCATACATTAT | | |
| | | | ACGAAGTTAT | | |

### DSD Shuffling Reaction

Cultures of DSD+pET28a empty vector (in vivo) or DSD+recombinase (in cognito) in E. coli DH5αPRO were made in 5 mL LB containing chloramphenicol (12.5 μg/mL) and kanamycin (50 μg/mL) and incubated overnight at 37° C. and 300 rpm. Next, cultures were diluted 1:10 in to 50 mL of LB containing chloramphenicol (12.5 μg/mL) and kanamycin (50 μg/mL) and grown to an OD₆₀₀ of ˜0.5-0.7 at 37° C. and 300 rpm. Subsequently, 15 mL of sample was removed for the 0 min induction time point and the rest were induced with anhydrotetracyline (aTc) at a final concentration of 100 ng/mL and incubated at 37° C. and 300 rpm, where 15 mL samples were removed at 60 and 120 min. Isolated samples were immediately pelleted and stored at −20° C. to prevent any further reactions from occurring. Plasmids were then column purified with Qiagen kits and stored in cell culture grade water (Cellgro) and diluted to 60 ng/μL and sent for sequencing. If required, plasmids were concentrated on an Eppendorf Vacufuge Plus. All experiments were performed in triplicate.

For DSD shuffling with Cre^{ts}, cultures of DSD-2α (in vivo) or DSD-2α+Cre^{ts} (in cognito) in E. coli DH5αPRO were made in 50 mL LB containing chloramphenicol (12.5 μg/mL) and for Cre^{ts} also carbenicillin (50 μg/mL), and incubated overnight at 30° C. and 300 rpm. Subsequently, 15 mL samples were removed for 0 min induction time point, and the cultures were then diluted 1:10 in 5 mL LB with antibiotics as above and induced with aTc (100 ng/mL), and incubated at 37° C. and 300 rpm for 6 hr. To cure out Cre^{ts}, cultures were then diluted 1:10000 in 50 mL of LB containing chloramphenicol (12.5 μg/mL) and incubated overnight at 42° C. and 300 rpm after which 15 mL samples were removed and sent for sequencing as above at a concentration of 30 ng/μL since this time there was only one plasmid present. To illustrate that Cre^{ts} was present when cultures were grown at 30° C. and absent after growth at 42° C., 100 ng of purified plasmids were run on a 1% agarose gel for 30 min at 130V on a Thermo Scientific Owl D2. All experiments were performed in triplicate.

To determine the distribution of the different states present after recombinase shuffling of the 2- and 4-state DSDs, 200 ng of shuffled samples from above were treated with 10 units of ClaI (NEB) and incubated at 37° C. for 4 hrs. Next, the digests were transformed in to E. coli TransforMax EPI300 cells (Epicentre) and plated on LB agar containing chloramphenicol (12.5 μg/mL) so that each cell would contain a single copy of DSDs. Then individual colonies were grown overnight in 8 mL of LB containing chloramphenicol (12.5 μg/mL) and 0.01% arabinose, to increase the plasmid yield from the DSD plasmids that were cloned in to pBAC-LacZ. Plasmids were then column purified with cell culture grade water (Cellgro) using Qiagen kits and sent for sequencing at GENEWIZ Inc. using the forward sequencing primer to determine the state of each DSD.

### Sanger Sequencing

All sequencing reactions were performed at GENEWIZ Inc. (Cambridge, Mass.) under blind experimental conditions. To ensure that there was no bias in the results reported, there was no contact made with the company prior, during, or after the experiments. All submitted samples were stored in cell culture grade water (Cellgro) and sequenced under ‘Difficult Template’ conditions to ensure there were no chemical interference with sequencing reactions and the most stringent conditions were used for each reaction. All forward sequencing reactions were performed with the primer—GACATTAACCTATAAAAATAGGC (SEQ ID NO: 7)—and all reverse sequencing reactions were performed with the primer—GCATCTTCCAGGAAATCTC (SEQ ID NO: 8). QS and CRL values were independently reported by Geneweiz Inc. Details for QS and CRL calculations, and their reflection on the quality of sequencing reads can be found at: www.genewiz.com. DNA sequencing data analysis, ClustalW alignments, and figure production were performed using Geneious Pro 5.5.8.

### Next Generation Sequencing

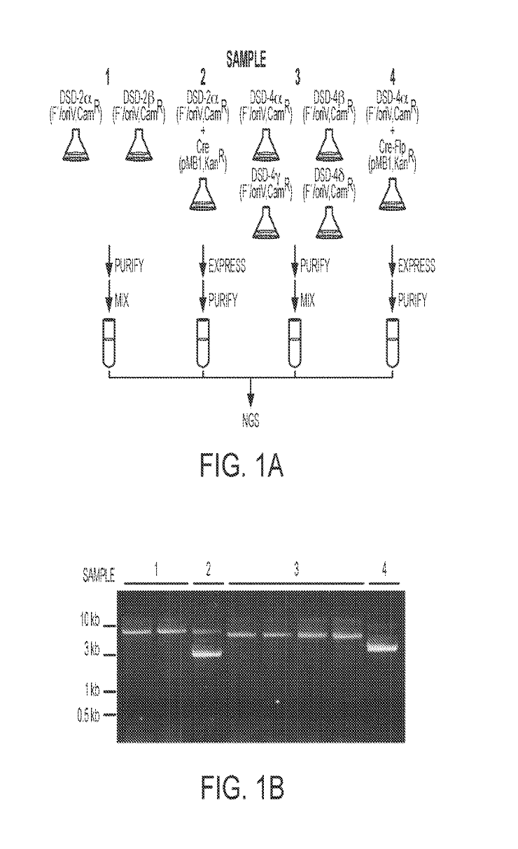

Samples submitted for NGS are described in FIGS. 1A-1B. For samples 1 and 3, plasmids were individually column purified with Qiagen kits and stored in cell culture grade water (Cellgro), and mixed at equal concentrations. Samples 2 and 4 were prepared as described above under in cognito conditions with recombinase induction for 120 min. 300 ng of purified plasmids were run on a 1% agarose gel for 30 min at 130V on a Thermo Scientific Owl D2 to verify purity. At least 300 ng of each sample were submitted to the MIT BioMicro Center (Cambridge, Mass.) for sequencing and annotation under blind experimental conditions. Samples 1-4 were used to produce libraries with a Nextera kit (Epicentre) followed by 1.5% agarose BluePippin (Sage Science) isolation of 450-800 bp inserts. Subsequently, libraries were pair-end sequenced using MiSeq (Illumina) run on a 600 nt v3 kit. NGS reads were then assembled in to contigs using Velvet, Trinity, and SOAP Denovo softwares, and subsequently further annotation was performed using the RAST annotation pipeline. To analyze the assembled scaffolds from SOAP Denovo assembly and RAST annotation, sequences were blasted against a plasmid database (http://plasmid.med.harvard.edu/) and hits with more than 90% sequence identity and a minimum of 100 bp alignment lengths were identified.

### Example 2: Steganography as a Platform for DNA Camouflage

Synthetic DNA has great utility for storing digital information. In nature, DNA has evolved as the storage medium of choice, where genetic information is stored, replicated, and communicated between cells and across species. Recent demonstrations of storing text, sound, and picture files in DNA have brought to light the potential of synthetic DNA to serve a role in long-term data storage and communication within a digital world. Compared to conventional optical and magnetic storage mediums, DNA has 1,000,000-fold higher storage capacity and can reliably store information for >2 million years, thus serving as a potential solution to concerns about future bit rot.

Several properties of DNA make it an attractive data storage medium: (i) chemical stability, (ii) lack of technological obsolescence, (iii) high density information storage, (iv) tolerance for harsh environmental conditions in spores, and (v) ease of replication. Accordingly, DNA is increasingly being utilized to store digital information for secure communications, watermarking of synthetic DNA, and archiving large texts. Such data may be confidential or of commercial value, thus necessitating new security measures.

As with optical and magnetic data storage, information security will be crucial for DNA data storage. To date, several methods of DNA security have been proposed. When information is stored in the physical architecture of self-assembling DNA molecules instead of the nucleotide sequence, data security can be implemented via programmed molecular self-assembly, akin to an encryption key. However, DNA self-assembly is not scalable for storing information beyond several bits. Alternatively, steganography—the science of storing information amongst other non-specific data—can be used to secure encoded information, where information encoding DNA strands are mixed with non-coding strands and data extraction relies on prior knowledge of the encoding algorithm. Finally, cryptographic methodologies such as one-time pads, AES or RSA algorithms can be adapted for DNA encoding to provide similarly high levels of security as achieved in conventional computer science approaches.

Cryptographic and steganographic methodologies have been proposed as safeguards against unauthorized individuals. Steganography and cryptography methods are advantageous for DNA security as they allow for facile scale-up and implementation into available DNA write and read technologies. To the best of Applicant's knowledge, there have not been any attempts at hindering the reading process-sequencing and bioinformatics analysis. Yet, obfuscating sequencing attempts would be most appropriate as a first line of defense, since DNA sequencing is the first step for data extraction.

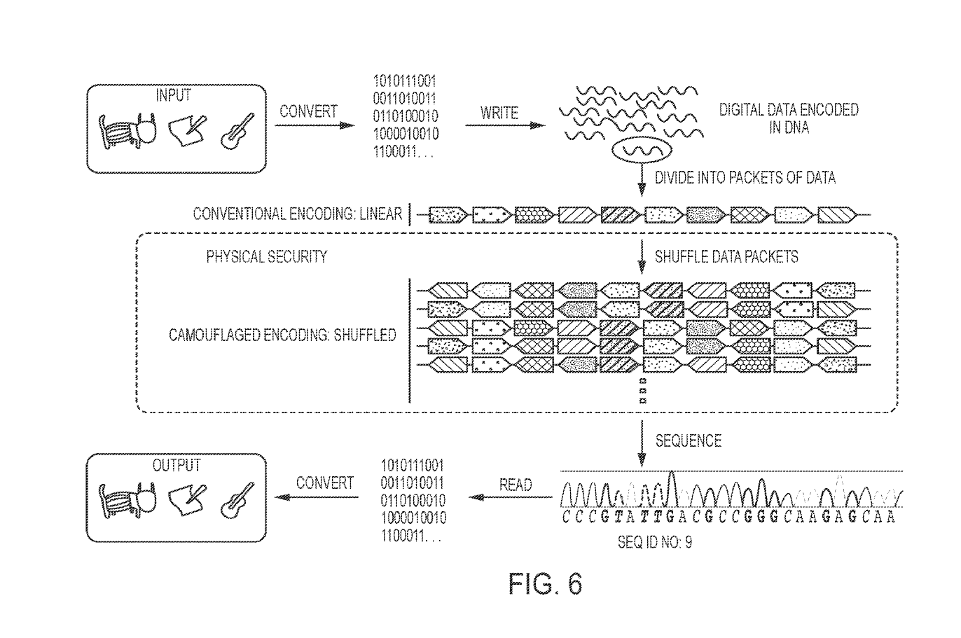

To write in DNA, a data file is first converted from the language of bits to the language of DNA, typically the nucleotides represented as A, C, G, and T. The corresponding DNA molecules are then produced by a combination of organic chemical synthesis and enzymatic assembly. This physical form of the information can be organized into packets reminiscent of the digital data packets used for digital communication (FIG. 6). To read, a sample of DNA molecules is subjected to sequencing, converting the encoded data from its molecular form back to a digital data file. While sequencing is the first step of data extraction, current security methods target the downstream decoding process. As a first layer of defense against unauthorized access, a physical security feature that would interfere with sequencing analysis was introduced, thereby camouflaging the data stored in DNA molecules.

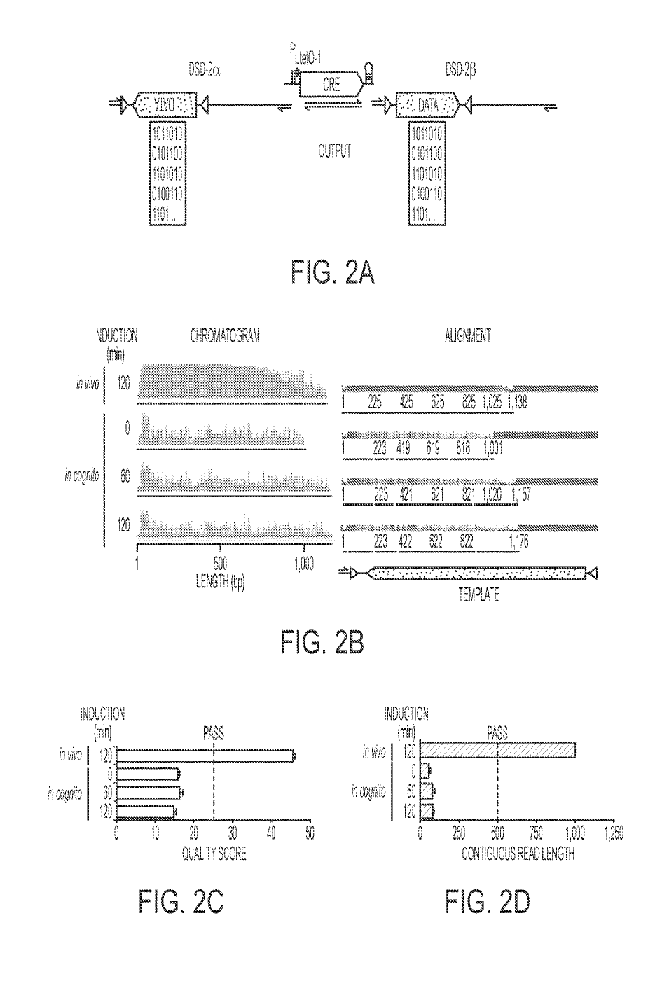

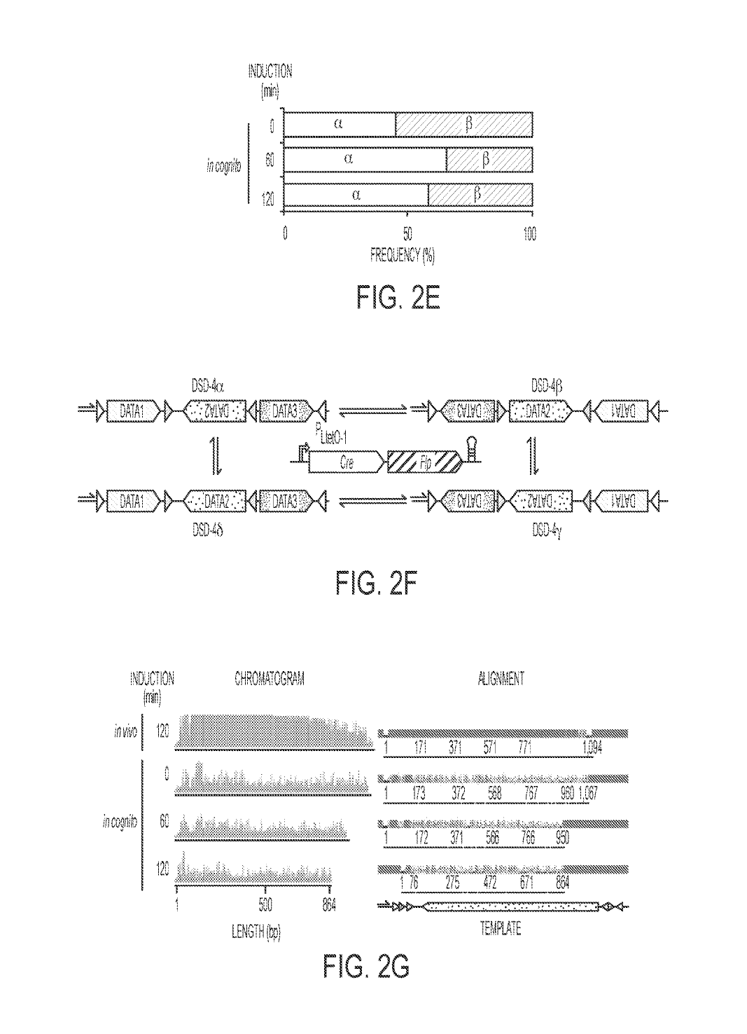

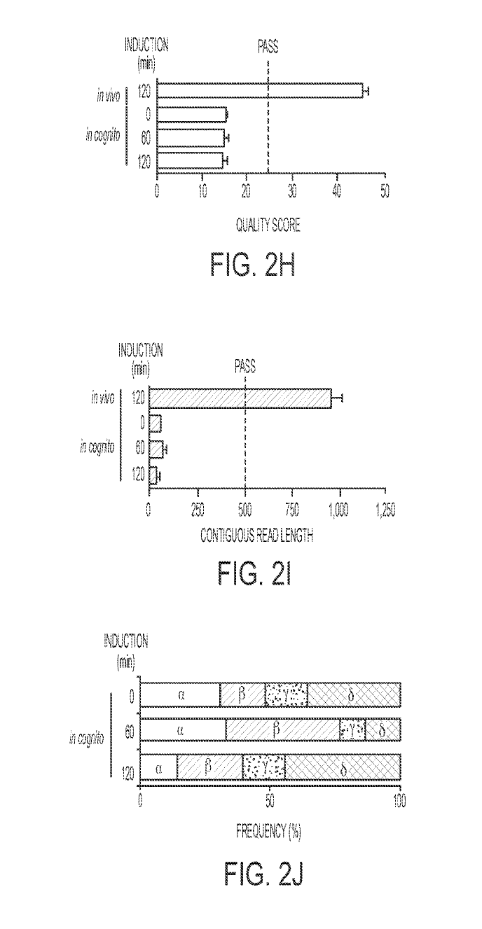

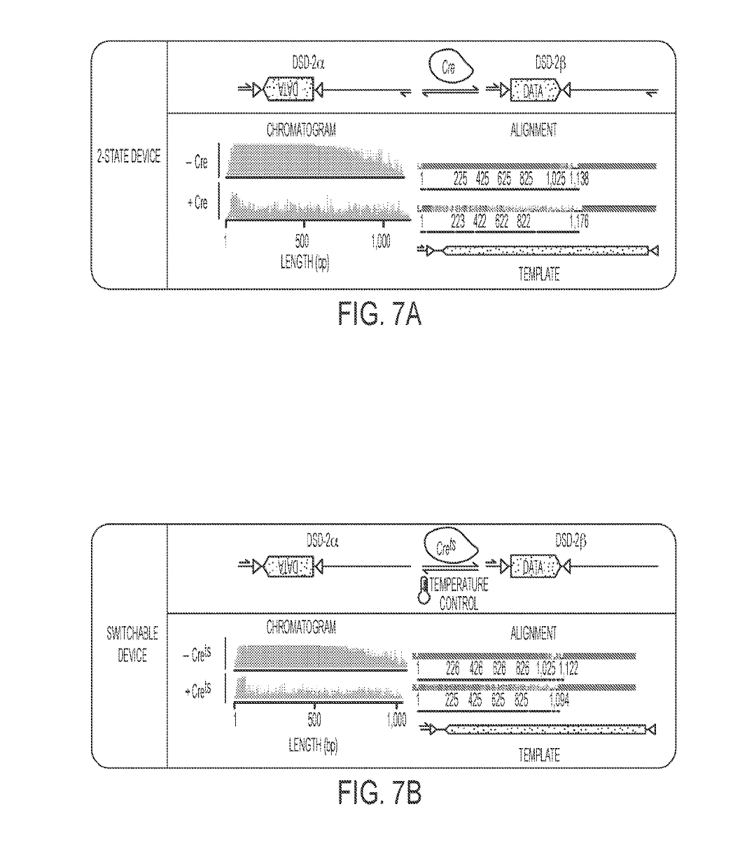

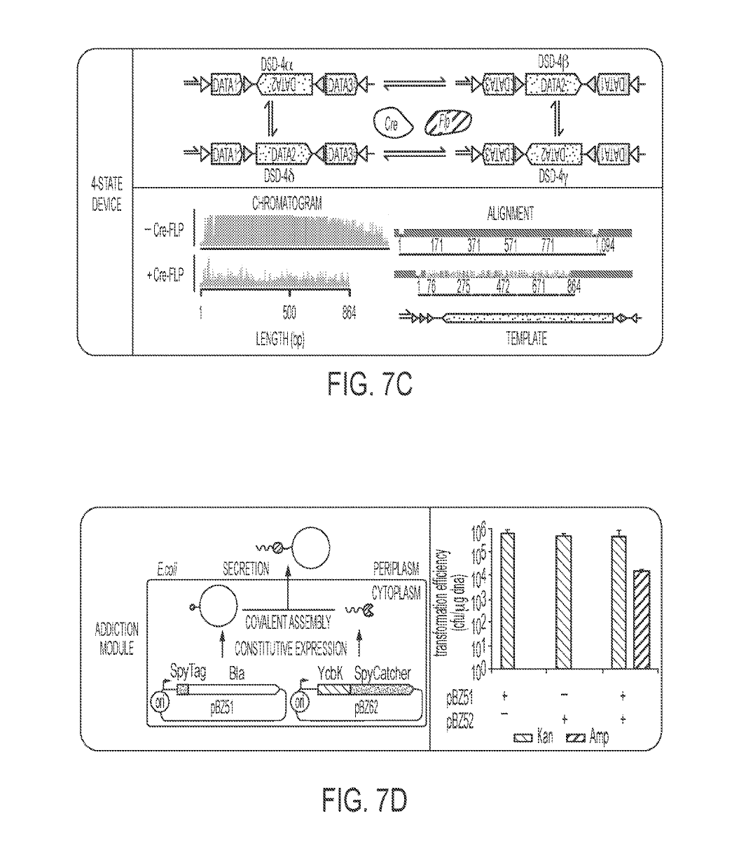

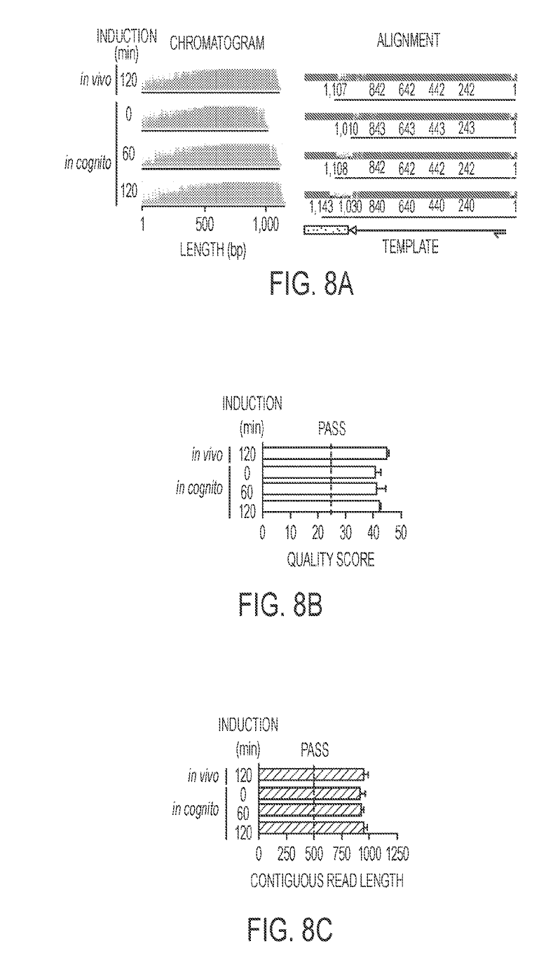

Cre is a bidirectional recombinase that flips DNA between two inverted loxP sites generating 2 states of the same sequence. Therefore, a 2-state DNA Steganographic Device (DSD-2) was developed by placing a 1.6 kb sequence between inverted loxP sites at single cellular copies (FIG. 2A and FIG. 7A). In the presence of Cre, the DSD would continuously oscillate between two possible states (α and β), while the embedded data would be unchanged (FIG. 7A). After DSD-2α was maintained in vivo, the DNA was extracted and sequenced resulting in a high quality chromatogram that aligned to the template (FIG. 2B). However, after DSD-2α was maintained in cognito, where Cre would randomly shuffle the DSD, the extracted DNA yielded poor sequencing reads that misaligned to the template (FIG. 8A). Furthermore, the quality score (QS) and contiguous read length (CRL) of the reads were significantly reduced and failed quality control measures (FIGS. 2C-2D and FIGS. 8B-8C). This camouflage effect was observed at 0, 60, and 120 min post Cre induction resulting in a random distribution of α and β states (FIG. 2E), likely a result of leaky expression. Cre was placed under P_{LtetO}-1 regulation that is tightly repressed in E. coli DH5αPRO generating one mRNA transcript per promoter every 10^{th} generation, which was sufficient to camouflage DNA.

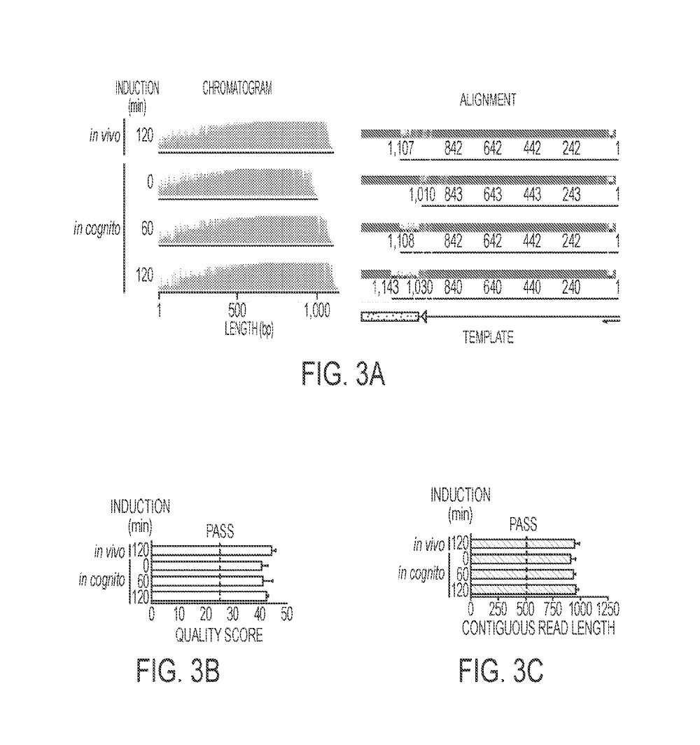

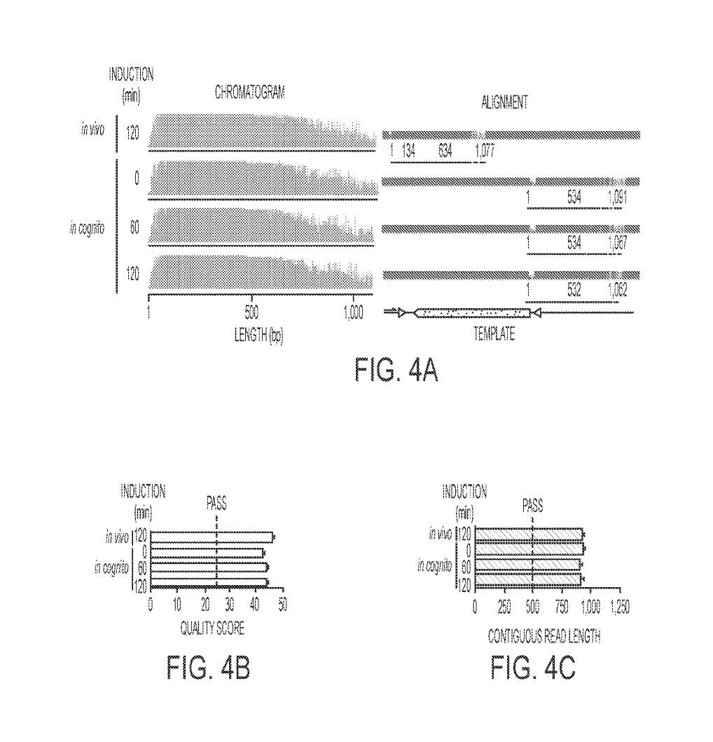

Shuffling of DSD-2α did not compromise DNA integrity as high quality sequencing reads were obtained under in vivo and in cognito conditions when extracted DNA was sequenced at 1 kb downstream of loxP (FIGS. 3A-3C). Interestingly, when DSD-2α was placed on a low copy plasmid with a p15A origin, it was excised under in cognito conditions, likely a result of trans-recombination events occurring between plasmids (FIG. 4).

For further complexity, a 4-state DSD was developed. The 4-state DSD is capable of camouflaging 3 DNA segments using Cre and Flp bidirectional recombinases to continuously oscillate segments between loxP and FRT sites, respectively (FIG. 2F and FIG. 7C). As expected, DSD-4α shuffling significantly compromised sequencing quality of in cognito samples producing low quality chromatograms that misaligned to the template at all time points tested, demonstrating that Cre and Flp efficiently function simultaneously at trace intracellular levels (FIG. 2G). Additionally, the QS and CRL values were greatly reduced and below acceptable levels (FIG. 2H-2I). These observations were a consequence of random DSD-4α shuffling by Cre and Flp, leading to an ever-changing cellular population harboring α, β, γ, and δ states (FIG. 2J). However, in 2% of the colonies analyzed the DSD was deleted leaving behind only a single loxP site, likely due to cross-reaction between recognition sites in different plasmids during cell division, where more than one copy may be present (FIG. 4).

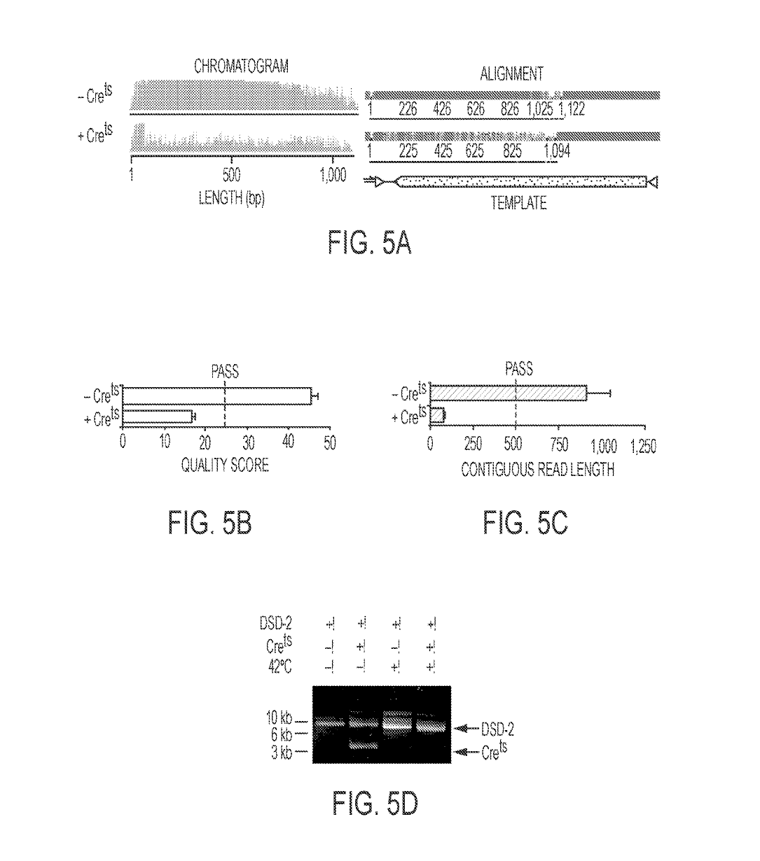

Next, the possibility of user-control over switching DNA maintained in vivo to in cognito was explored. Even the most efficient gene repression technologies can still lead to minute levels of transcription, which can be detrimental when maintaining cells for many generations in the presence of recombinases. Therefore, Cre was placed on a temperature sensitive plasmid (Cre^{ts}) to allow users to camouflage DSDs harbored in cells, and then remove Cre^{ts} without leaving a trace (FIG. 7B). When tested with DSD-2α, this resulted in poor quality sequencing data and since Cre^{ts} was cured out of the cells, there was no evidence left behind (FIG. 5).

Although steganography has been demonstrated as a viable platform for protecting information on DNA against Sanger sequencing by unauthorized individuals, one may also attempt to analyze unknown DNA with more powerful Next Generation Sequencing (NGS) technologies. Therefore, the ability of a third party to provide a the final annotated and assembled sequence of camouflaged DNA samples was tested in a blind experiment.

Four samples with DSD-2 and DSD-4 templates in dH₂O were prepared and submitted to an outside party for NGS analysis without providing any information about the samples. The four samples sent for NGS were: (1) Sample 1: DSD-2[α+β], (2) Sample 2: DSD-2[α]+Cre, (3) Sample 3: DSD-4[α+β+γ+δ], and (4) Sample 4: DSD-4[α]+Cre-Flp. The outside party was requested to assemble the DNA sequence of each sample (FIG. 1A). Although all samples successfully produced over 2 million reads with the correct GC content (Table 2), samples 1 and 3 posed the most difficulty in library preparation due to the blind nature of the experiment. Following de novo assembly of the NGS reads, the four samples provided vastly different data even though the DNA contents were highly similar (Table 3). It was surprising to find that samples 1 and 3 demonstrated the most complexity, even though they were simpler than 2 and 4 at the sequence level.

Subsequently, the millions of reads produced from each sample were assembled to generate between 248 and 711 scaffolds with greatly different contig sizes. When the scaffolds were blasted against a plasmid database as no template information was provided, only a small percentage of the scaffolds were able to align to database sequences (Table 4). To identify hits during BLAST analysis, more that 90% sequence identity and a minimum of 100 bp alignment length was required. Only 1-2% of the assembled scaffolds could be aligned to a maximum of 4 plasmids in the database. At the conclusion of a month of analysis, the outside party was unable to provide the annotated and assembled sequences of the provided samples. This experiment demonstrated that while NGS is a powerful analytical tool, it would be difficult for unauthorized individuals to determine the sequences of camouflaged DNA samples. This scenario would become even more difficult if the DNA harbors digital data that is encrypted rather than genetic information that is simpler to analyze with bioinformatics tools, where genetic elements would be easier to identify.

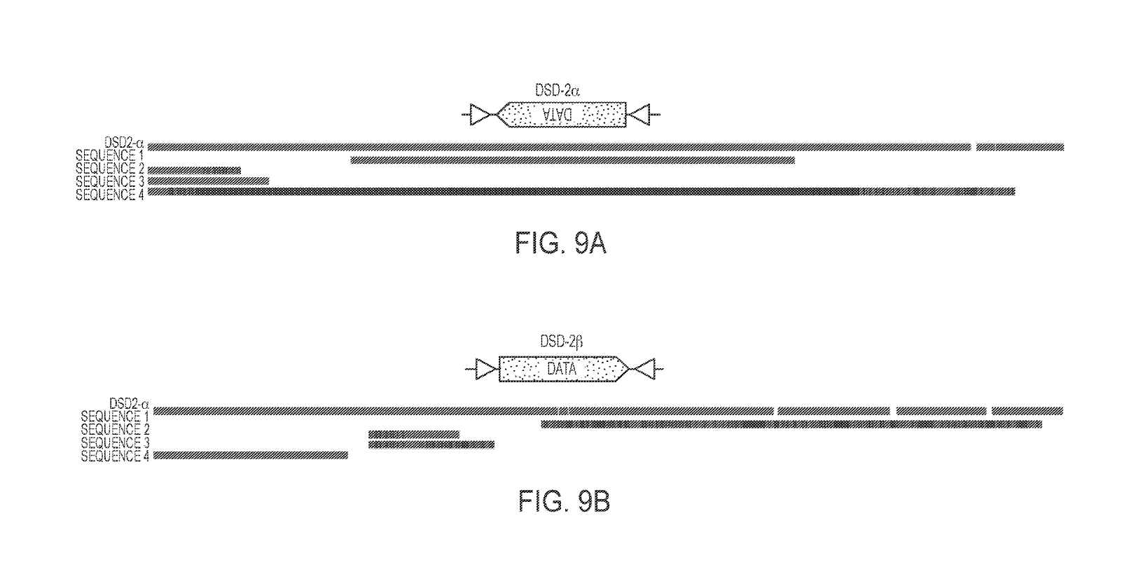

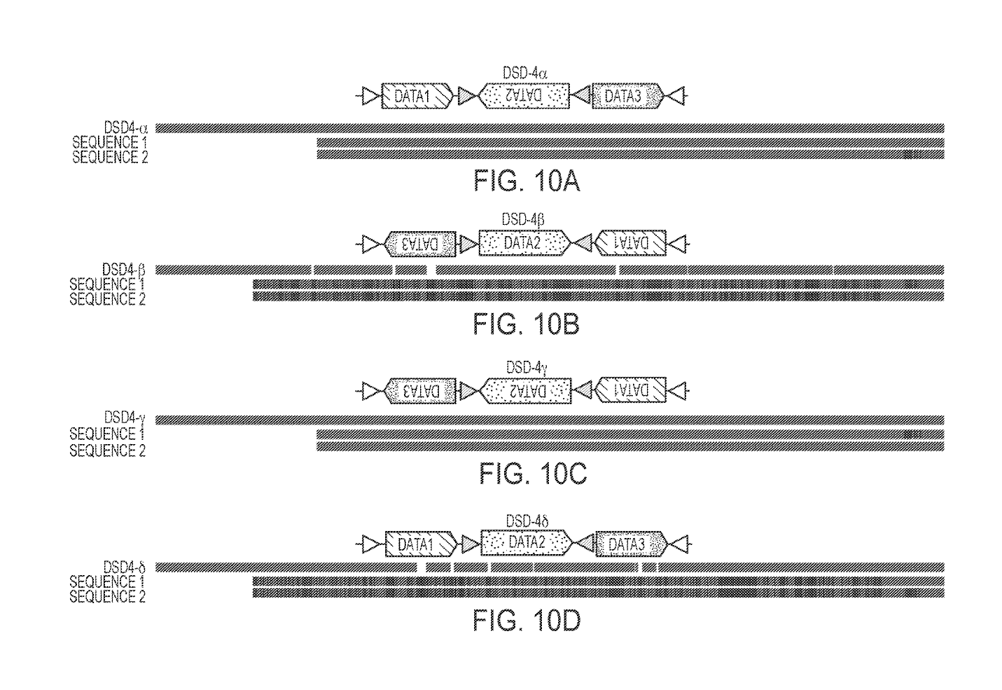

Subsequently, the sequencing service was informed that the samples contained purified plasmids encoding digital data and provided the sequence of the backbone vectors (but not the number of different plasmids present per sample). The sequencing service was then asked to provide the sequence of the data packets present in the samples. Interestingly, the outside party did not identify any data sequences in samples 2 and 4, however they were able to identify several data sequences for samples 1 and 3 (Table 5). Alignment of the NGS identified data sequences to the DNA templates revealed partial sequences were reliably identified for both the DSD-2 and DSD-4 samples (FIGS. 9A-9B and FIGS. 10A-10D). However, when the plasmid sequence and the total number of plasmids are known, then almost complete data recovery can be performed. Therefore, DNA shuffling can be used to hinder data comprehension via NGS. However, with prior knowledge of the contents the data can be reliably extracted.

Recombinase shuffling of DSD devices in multi-copy plasmids leads to data excision (FIG. 5). Therefore, we sought to develop a method of maintaining DSD-2[α] and DSD-2[β] states stably within the same cell, so that bacterial transformation of any individual state will not lead to viable cells, but co-transformation will produce viable colonies that also maintain both α and β states.

Two plasmids with the same origin of replication and selection marker cannot be stably maintained within a single cell over extended time periods, because as cells reproduce, the absence of specific positive selection leads to segregational loss. To enable dual plasmid maintenance, the splitting of one selection marker between 2 plasmids that possess the same origin of replication was investigated. Therefore, cell survival is based on co-dependency on 2 plasmids, which is referred to as an“addiction module”.

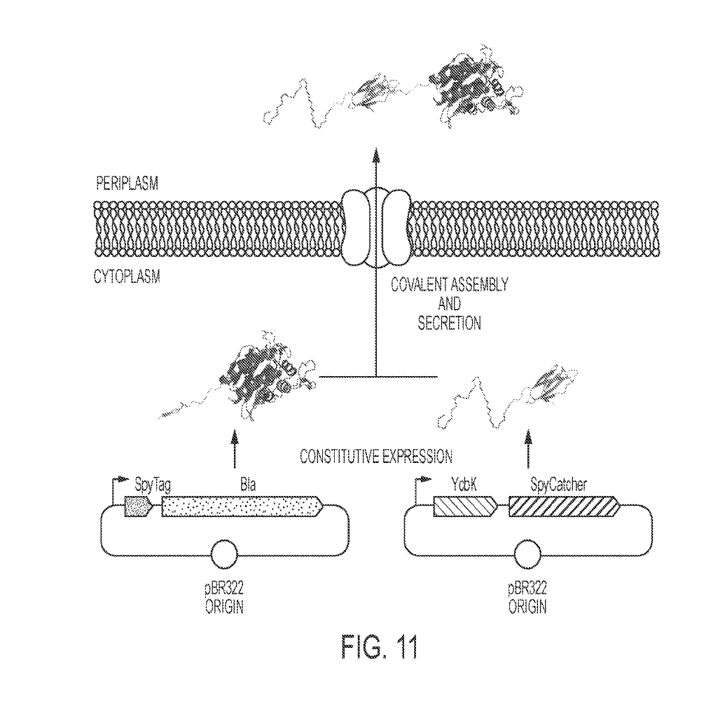

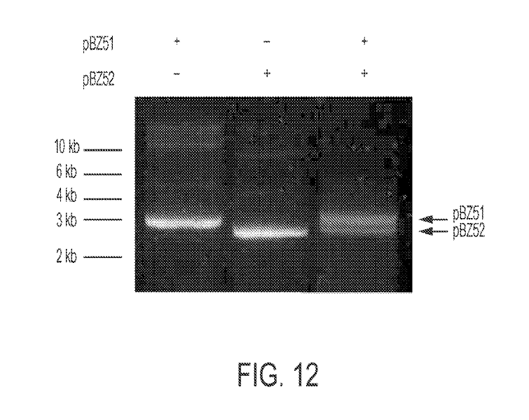

To develop an addiction module, programmable isopeptide-based post-translational protein assembly was utilized. For example, SpyTag and SpyCatcher can be fused to the terminal ends of the β-lactamase (Bla) gene (encoding ampicillin resistance) to circularize the enzyme for enhanced stability. However, this construct lacked a signal sequence for periplasmic secretion that is required for imparting bacterial resistance. Accordingly, SpyTag/SpyCatcher was utilized to attach a secretion tag to Bla, thereby requiring a two-component process for achieving resistance (FIG. 11). To enable secretion, we utilized the twin-arginine translocation (Tat) pathway that allows for the secretion fully folded protein complexes, attaching SpyTag to Bla and the Tat secretion tag YcbK to SpyCatcher. In this addiction module, SpyTag-Bla and YcbK-SpyCatcher are constitutively expressed from two separate plasmids, where transformation of either plasmid individually cannot provide resistance but dual transformation with both plasmids allows survival under selection. To test the addiction module, SpyTag-Bla in pBZ51 and YcbK-SpyCatcher were cloned in pBZ52, where both plasmids encoded the same origin of replication and also a kanamycin resistance marker as a control. When selected on kanamycin, individual and dual transformation of pBZ51 and pBZ52 was efficient and produced similarly high levels of colonies (FIG. 8D). However, when selected on selective media (e.g., carbenecillin or ampicillin), separate transformations with pBZ51 or pBZ52 did not yield any colonies. Dual transformation with a mixture of both pBZ51 and pBZ52 produced many colonies when selected on ampicillin, indicating the addiction module is able to reconstitute resistance via programmed post-translational protein assembly. Extraction from a single colony yielded both plasmids, as analyzed on an agarose gel, demonstrating that the two plasmids were stably maintained together (FIG. 12).

The DNA storage of increasingly valuable information and economic assets necessitates new bio-safeguards that provide additional protection extending beyond legal measures. Here, we have briefly demonstrated the use of steganography to introduce sequence complexity to a population without affecting the stored information; thereby serving to camouflage DNA segments against sequencing by unauthorized individuals. This principle can be extended beyond living cells, and can be applied during DNA synthesis as bio-safeguards.

TABLE 2
Raw data produced from NGS analysis of samples 1-4.

| Sample | 1 | 2 | 3 | 4 |
|---|---|---|---|---|
| Total Sequences | 2,035,696 | 2,827,422 | 3,762,818 | 2,665,635 |
| % GC | 48 | 49 | 47 | 46 |

TABLE 3
NGS sequencing statistics of assembled data for
samples 1-4 under blind experimental conditions.

| Sample | 1 | 2 | 3 | 4 |
|---|---|---|---|---|
| Sequence size | 4,484,782 | 109,143 | 4,575,261 | 238,314 |
| Number of scaffolds | 711 | 248 | 500 | 536 |
| % GC | 50.7 | 49.3 | 50.7 | 50.1 |
| Shortest contig size | 301 | 300 | 306 | 300 |
| Median sequence | 3,897 | 360 | 3,943 | 390 |
| size | | | | |
| Mean sequence size | 6,307.7 | 440.1 | 9,150.5 | 444.6 |
| Longest contig size | 51,023 | 5,385 | 93,737 | 5,397 |
| Number of | 564 | 2 | 576 | 2 |
| subsystems | | | | |
| Number of coding | 4,300 | 64 | 4,410 | 190 |
| sequences | | | | |
| Number of RNAs | 34 | 0 | 30 | 0 |

TABLE 4
Identification of annotated and assembled samples
1-4 by BLAST analysis against a plasmid database.

| | Total | Aligned | | |
|---|---|---|---|---|
| Sample | Scaffolds | Scaffolds | % Aligned | identified Vectors |
| 1 | 711 | 12 | 1.7 | pBluescriptR (Amp^{R}) |
| | | | | pDONR221 (Kan^{R}) |
| | | | | pOTB7 (Cam^{R}) |
| 2 | 248 | 3 | 1.2 | pBluescriptR (Amp^{R}) |
| | | | | pDONR221 (Kan^{R}) |
| | | | | pOTB7 (Cam^{R}) |
| 3 | 500 | 10 | 2.0 | pBluescriptR (Amp^{R}) |
| | | | | pDONR221 (Kan^{R}) |
| | | | | pOTB7 (Cam^{R}) |
| 4 | 536 | 6 | 1.1 | pBluescriptR (Amp^{R}) |
| | | | | pDONR221 (Kan^{R}) |
| | | | | pOTB7 (Cam^{R}) |
| | | | | pK7-GFP (Amp^{R}) |

TABLE 5

| | Sequence | | SEQ ID |
|---|---|---|---|
| Sample | Number | Identified Insert | NO: |
| 1 | 1 | TTCATCCATGCCATGTGTAATCCCAGCAGCTGTTAC | 10 |
| | | AAACTCAAGAAGGACCATGTGGTCTCTCTTTTCGTT | |
| | | GGGATCTTTCGAAAGGGCAGATTGTGTGGACAGGT | |
| | | AATGGTTGTCTGGTAAAAGGACAGGGCCATCGCCA | |
| | | ATTGGAGTATTTTGTTGATAATGGTCTGCTAGTTGA | |
| | | ACGCTTCCATCTTCAATGTTGTGTCTAATTTTGAAG | |
| | | TTAACTTTGATTCCATTCTTTTGTTTGTCTGCCATGA | |
| | | TGTATACATTGTGTGAGTTATAGTTGTATTCCAATT | |
| | | TGTGTCCAAGAATGTTTCCATCTTCTTTAAAATCAA | |
| | | TACCTTTTAACTCGATTCTATTAACAAGGGTATCAC | |
| | | CTTCAAACTTGACTTCAGCACGTGTCTTGTAGTTCC | |
| | | CGTCATCTTTGAAAAATATAGTTCTTTCCTGTACAT | |
| | | AACCTTCGGGCATGGCACTCTTGAAAAAGTCATGC | |
| | | TGTTTCATATGATCTGGGTATCTCGCAAAGCATTGA | |
| | | ACACCATAACCGAAAGTAGTGACAAGTGTTGGCCA | |
| | | TGGAACAGGTAGTTTTCCAGTAGTGCAAATAAATT | |
| | | TAAGGGTAAGTTTTCCGTATGTTGCATCACCTTCAC | |
| | | CCTCTCCACTGACAGAAAATTTGTGCCCATTAACA | |
| | | TCACCATCTAATTCAACAAGAATTGGGACAACTCC | |
| | | AGTGAAAAGTTCTTCTCCTTTACGCATGGTATATCT | |
| | | CCTTCTTAAAGTGGTCAGTGCGTCCTGCTGATGTGC | |
| | | TCAGTATCTTGTTATCCGCTCACAATGTAAATTG | |
| | 2 | ATAACTTCGTATAATGTATGCTATACGAAGTTATG | 11 |
| | | CAGTTTCATTTGATGCTCGATGAGTTTTTCTAAGAA | |
| | | TTAATTCATGAGCGGATACAATTGTGAGCGGATAA | |
| | | CAATTTACATTGTGAGCGGATAACAAGATACTGAG | |
| | | CACATCAGCAGGACGCACTGACC | |
| | 3 | ATAACTTCGTATAATGTATGCTATACGAAGTTATG | 12 |
| | | CAGTTTCATTTGATGCTCGATGAGTTTTTCTAAGAA | |
| | | TTAATTCATGAGCGGATACATATTTGAATGTATTTA | |
| | | GAAAAATAAACAAATAGGGGTTCCGCGCACATTTC | |
| | | CCCGAAAAGTGCCACCTAGGTATCTGGCACTACGT | |
| | | TCAGGTAACCTGAAGCTCGAATCCAGTACTCGACG | |
| | | TC | |
| | 4 | ATAACTTCGTATAATGTATGCTATACGAAGTTATG | 13 |
| | | CTAGCTATGGAGAAACAGTAGAGAGTTGCGATAA | |
| | | AAAGCGTCAGGTAGGATCCGCTAATCTTATGGATA | |
| | | AAAATGCTATGGCATAGCAAAGTGTGACGCCGTGC | |
| | | AAATAATCAATGTGGACTTTTCTGCCGTGATTATA | |
| | | GACACTTTTGTTACGCGTTTTTGTCATGGCTTTGGT | |
| | | CCCGCTTTGTTACAGAATGCTTTTAATAAGCGGGG | |
| | | TTACCGGTTTGGTTAGCGAGAAGAGCCAGTAAAAG | |
| | | ACGCAGTGACGGCAATGTCTGATGCAATATGGACA | |
| | | ATTGGTTTCTTCGTCGGTCTGATTATTAGTCTGGG | |
| 2 | No insert sequence identified | | |
| 3 | 1 | TTCATCCATGCCATGTGTAATCCCAGCAGCTGTTAC | 14 |
| | | AAACTCAAGAAGGACCATGTGGTCTCTCTTTTCGTT | |
| | | GGGATCTTTCGAAAGGGCAGATTGTGTGGACAGGT | |
| | | AATGGTTGTCTGGTAAAAGGACAGGGCCATCGCCA | |
| | | ATTGGAGTATTTTGTTGATAATGGTCTGCTAGTT | |
| | | GAACGCTTCCATCTTCAATGTTGTGTCTAATTTTGA | |
| | | AGTTAACTTTGATTCCATTCTTTTGTTTGTCTGCCAT | |
| | | GATGTATACATTGTGTGAGTTATAGTTGTATTCCAA | |
| | | TTTGTGTCCAAGAATGTTTCCATCTTCTTTAAAATC | |
| | | AATACCTTTTAACTCGATTCTATTAACAAGGGTATC | |
| | | ACCTTCAAACTTGACTTCAGCACGTGTCTTGTAGTT | |
| | | CCCGTCATCTTTGAAAAATATAGTTCTTTCCTGTAC | |
| | | ATAACCTTCGGGCATGGCACTCTTGAAAAAGTCAT | |
| | | GCTGTTTCATATGATCTGGGTATCTCGCAAAGCATT | |
| | | GAACACCATAACCGAAAGTAGTGACAAGTGTTGGC | |
| | | CATGGAACAGGTAGTTTTCCAGTAGTGCAAATAAA | |
| | | TTTAAGGGTAAGTTTTCCGTATGTTGCATCACCTTC | |
| | | ACCCTCTCCACTGACAGAAAATTTGTGCCCATTAA | |
| | | CATCACCATCTAATTCAACAAGAATTGGGACAACT | |
| | | CCAGTGAAAAGTTCTTCTCCTTTACGCATGGTATAT | |
| | | CTCCTTCTTAAAGTGGTCAGTGCGTCCTGCTGATGT | |
| | | GCTCAGTATCTTGTTATCCGCTCACAATGTAAATTG | |
| | | TTATCCGCTCACAATTGTATCCGCTCATGAATTAAT | |
| | | TCTTAGAAGTTCCTATACTTTCTAGAGAATAGGAA | |
| | | CTTCAGGCATTGATGGAATCGTAGTCTCAATAACT | |
| | | TCGTATAGCATACATTATACGAAGTTAT | |
| | 2 | TTCATCCATGCCATGTGTAATCCCAGCAGCTGTTAC | 15 |
| | | AAACTCAAGAAGGACCATGTGGTCTCTCTTTTCGTT | |
| | | GGGATCTTTCGAAAGGGCAGATTGTGTGGACAGGT | |
| | | AATGGTTGTCTGGTAAAAGGACAGGGCCATCGCCA | |
| | | ATTGGAGTATTTTGTTGATAATGGTCTGCTAGTTGA | |
| | | ACGCTTCCATCTTCAATGTTGTGTCTAATTTTGAAG | |
| | | TTAACTTTGATTCCATTCTTTTGTTTGTCTGCCATGA | |
| | | TGTATACATTGTGTGAGTTATAGTTGTATTCCAATT | |
| | | TGTGTCCAAGAATGTTTCCATCTTCTTTAAAATCAA | |
| | | TACCTTTTAACTCGATTCTATTAACAAGGGTATCAC | |
| | | CTTCAAACTTGACTTCAGCACGTGTCTTGTAGTTCC | |
| | | CGTCATCTTTGAAAAATATAGTTCTTTCCTGTACAT | |
| | | AACCTTCGGGCATGGCACTCTTGAAAAAGTCATGC | |
| | | TGTTTCATATGATCTGGGTATCTCGCAAAGCATTGA | |
| | | ACACCATAACCGAAAGTAGTGACAAGTGTTGGCCA | |
| | | TGGAACAGGTAGTTTTCCAGTAGTGCAAATAAATT | |
| | | TAAGGGTAAGTTTTCCGTATGTTGCATCACCTTCAC | |
| | | CCTCTCCACTGACAGAAAATTTGTGCCCATTAACA | |
| | | TCACCATCTAATTCAACAAGAATTGGGACAACTCC | |
| | | AGTGAAAAGTTCTTCTCCTTTACGCATGGTATATCT | |
| | | CCTTCTTAAAGTGGTCAGTGCGTCCTGCTGATGTGC | |
| | | TCAGTATCTTGTTATCCGCTCACAATGTAAATTGTT | |
| | | ATCCGCTCACAATTGTATCCGCTCATGAATTAATTC | |
| | | TTAGAAGTTCCTATACTTTCTAGAGAATAGGAACT | |
| | | TCCTCATCGAGCATCAAATGAAACTGCATAACTTC | |
| | | GTATAGCATACATTATACGAAGTTAT | |

| | |
|---|---|
| 4 | No insert sequence identified |

What is claimed is:

1. A method for camouflaging a nucleic acid sequence comprising:
preparing a pool of recombinant nucleic acid constructs, wherein at least one of the constructs comprises the nucleic acid sequence to be camouflaged and,
wherein the pool is heterogeneous with respect to the orientation of the nucleic acid sequence to be camouflaged.

2. The method of claim 1, wherein the pool of constructs is obtained by nucleic acid synthesis.

3. The method of claim 1, wherein the pool of constructs is maintained in a cell or cells.

4. The method of claim 1, wherein the pool of constructs is maintained in a non-cellular environment.

5. A method for camouflaging a nucleic acid sequence comprising
preparing a recombinant nucleic acid construct that comprises the nucleic acid sequence to be camouflaged, wherein the nucleic acid sequence is flanked by opposing recognition sites of a bidirectional recombinase;
introducing the recombinant nucleic acid construct into a cell that comprises the bidirectional recombinase;
culturing the cell under conditions in which the cell multiplies to produce a plurality of cells;
creating a population of cells that is heterogeneous with respect to the orientation of the nucleic acid sequence by culturing the plurality of cells under conditions in which the bidirectional recombinase is expressed, whereby the nucleic acid sequence flanked by the opposing recognition sites of the bidirectional recombinase is inverted in a portion of the plurality of cells, thereby camouflaging the nucleic acid sequence.

6. A method for camouflaging a nucleic acid sequence comprising:
(a) preparing a recombinant nucleic acid construct that comprises the nucleic acid sequence to be camouflaged, wherein the nucleic acid sequence is flanked by opposing recognition sites of a bidirectional recombinase;
(b) combining a plurality of the construct of (a) and a functional bidirectional recombinase in vitro, whereby the nucleic acid sequence flanked by the opposing recognition sites of the bidirectional recombinase is inverted, thereby producing a heterogeneous population of camouflaged constructs; and,
(c) maintaining the heterogeneous population of (b) in a non-cellular environment.

7. The method of any of claims 1 to 6, wherein the nucleic acid sequence is dispersed across a plurality of nucleic acids or comprises of a plurality of segments.

8. The method of claim 7, wherein at least two of the plurality of nucleic acids or the plurality of segments are flanked by opposing recognition sites of at least two different bidirectional recombinases, and wherein the cell or plurality of cells comprises the at least two different bidirectional recombinases.

9. The method of any one of claims 5 to 8, wherein the bidirectional recombinase(s) is/are expressed in the cell on one or more temperature sensitive plasmids.

10. The method of any one of claims 5 to 8, wherein the bidirectional recombinase(s) is/are expressed constitutively in the plurality of cells.

11. The method of any one of claims 5 to 8, wherein the bidirectional recombinase(s) is/are expressed under control of one or more conditional promoters in the plurality of cells.

12. The method of any one of claims 5 to 11, wherein the cell is a prokaryotic cell.

13. The method of any one of claims 5 to 11, wherein the cell is a eukaryotic cell.

14. The method of any one of claims 5 to 13, wherein the recombinant nucleic acid construct is integrated into the genome of the plurality of cells.

15. The method of any one of claims 5 to 14, wherein the cell(s) comprises at least one of Cre, Flp and R bidirectional recombinases and wherein the recognition sites flanking the nucleic acid sequence are operable with the at least one bidirectional recombinases and are selected from loxP, FRT and RS recognition sites.

16. The method of claim 15, wherein the cell comprises two or more bidirectional recombinases and wherein the recognition sites flanking the nucleic acid are operable with the two or more bidirectional recombinases.

17. The method of any one of claims 5 to 14, wherein the cell(s) comprises at least one unidirectional recombinase and wherein the recognition sites flanking the nucleic acid sequences are operable with the at least one unidirectional recombinase.

18. The method of any one of claims 5 to 17, wherein the nucleic acid sequence is encrypted.

19. A method of securely transmitting information to a recipient that is encoded in a nucleic acid sequence comprising
providing to a recipient a population of cells comprising a camouflaged nucleic acid sequence according to the methods of any one of claims 5 to 18.

20. A method of securely transmitting information to a recipient that is encoded in a nucleic acid sequence comprising
providing to a recipient a camouflaged nucleic acid construct according to the methods of any of the preceding claims.

21. The method of claim 19 or claim 20, wherein the recipient determines the sequence of the camouflaged nucleic acid sequence.

22. The method of any one of claims 19-21, wherein the nucleic acid sequence is encrypted.
